(19) United States
(12) Patent Application Publication
(10) Pub. No.: US 2022/0204465 A1
(43) Pub. Date: Jun. 30, 2022

(54) PROCESS FOR THE CARBONYLATION OF EPOXIDES

(71) Applicant: Covestro Intellectual Property GmbH & Co. KG, Leverkusen (DE)

(72) Inventors: Jens Langanke, Mechernich (DE); Matthias Leven, Köln (DE); Christoph Guertler, Köln (DE); Suresh Raju, Aachen (DE)

(21) Appl. No.: 17/607,737

(22) PCT Filed: Jun. 5, 2020
(86) PCT No.: PCT/EP2020/065574

§ 371 Date: Oct. 29, 2021

(30) Foreign Application Priority Data
Jun. 12, 2019 (EP) 19179686.1

(51) Int. Cl. C07D 305/12; C08G 63/08; C08G 63/82; B01J 31/02; B01J 31/18; B01J 31/22

(52) U.S. Cl. CPC C07D 305/12; C08G 63/08; C08G 63/823; B01J 2531/64; B01J 31/183; B01J 31/2213; B01J 31/2243; B01J 31/0265

(57) ABSTRACT

A process for the carbonylation of epoxides in the presence of catalyst systems, wherein the carbonylation takes place in the presence of carbon monoxide, and wherein the catalyst system contains a molybdenum-based compound. Carbonylation products as well as carbonylation derivatives and to the use of the claimed catalyst systems for the carbonylation of epoxides are also provided.

### CROSS REFERENCE TO RELATED APPLICATIONS

This application is a U.S. national stage application, filed under 35 U.S.C. § 371, of International Application No. PCT/EP2020/065574, which was filed on Jun. 5, 2020, and which claims priority to European Patent Application No. 19179686.1 which was filed on Jun. 12, 2019. The contents of each are hereby incorporated by reference into this specification.

### FIELD

The invention relates to a process for the carbonylation of epoxides in the presence of catalyst systems, wherein the carbonylation takes place in the presence of carbon monoxide, and wherein the catalyst system comprises a molybdenum-based compound. The invention further relates to carbonylation products and carbonylation conversion products and to the use of catalyst systems according to the invention for carbonylation of epoxides.

### BACKGROUND

DE 10235316 A1 discloses a process for preparing lactones by catalytic carbonylation of oxiranes, in which a catalyst system is used composed of a) at least one carbonylation catalyst A composed of neutral or anionic transition metal complexes of metals from groups 5 to 11 of the Periodic Table of the Elements and b) at least one chiral Lewis acid B as catalyst, with the exception of [(salph)Al(THF)2][Co(CO)4].

WO 2006/058681 A2 describes a process for preparing enantiomerically enriched lactones by catalytic carbonylation of lactones to anhydrides in the presence of a neutral or anionic transition metal complex.

WO 2016/015019 A1 describes a process for preparing aluminum complexes, which are used to catalyze the carbonylation of epoxides.

JP2013173090 describes the carbonylation of epoxides using immobilized catalyst systems.

EP 0176370 A1 describes a process for preparing lactones via the cyclizing esterification of unsaturated alcohols with carbon monoxide in the presence of a catalyst. Here, Mo is described as component of a bi-metallic catalyst system. beta-Lactones, i.e. cyclic 4-ring esters, are neither described nor claimed. Also epoxides as possible substrates and/or intermediates are neither described nor claimed.

WO 2011/163309 A2 describes a two-stage process for producing polyhydroxybutyrate and/or polyhydroxypropionate homopolymers with subsequent thermal decomposition to form unsaturated acids such as crotonic acid and/or acrylic acid. In the first stage, epoxides are carbonylated to produce the corresponding beta-lactones and in the second stage converted to the corresponding homopolymers.

Cobalt and molybdenum compounds differ significantly in their regulatory classification. The US federal authority OSHA stipulates a time-weighted average value for occupational exposure limits of ≤0.1 ppm for cobalt carbonyls and cobalt hydrocarbonyls (cf. Clinical Toxicology, 1999, 37, 201-216), whereas a time-weighted average value for exposure limits for soluble molybdenum compounds is 5 ppm and for insoluble molybdenum compounds is 10 ppm (cf. Clinical Toxicology, 1999, 37, 231-237).

The global production of cobalt in 2016 was 123 000 tons (according to the Department of Natural Resources, Canada). Whereas the global production of molybdenum in 2007 was ca. 211 000 tons (cf. statistics of the United States Geological Society). In the course of 2018, the price was quoted at $25/kg on the commodity market (see London Metal Exchange, historical price data for molybdenum).

### SUMMARY

Starting from the prior art, the object of the present invention was to redress the disadvantages of cobalt-based catalysts for the carbonylation of epoxides.

The object of the present invention was therefore to provide a simplified process for the carbonylation of epoxides to form carbonylation products using a catalyst system, wherein in particular beta-lactones, cyclic ester ether compounds and, as conversion products thereof, polyhydroxyalkanoate (co)polymers, especially polyhydroxybutyrate and/or polyhydroxypropionate (co) polymers, should be provided by a direct and one-step process.

In this case, a chemically more stable catalyst system should be used, so that longer storage and/or technically simpler storage, handling and use compared to the cobalt-based systems described above from the prior art is possible. In the cobalt-based systems in the prior art described above, cobalt is in the very sensitive oxidation state of −1 and can be degraded exceptionally easily with oxidants of all kinds, as well as hydroxyl compounds or water. In addition, the catalyst systems according to the invention should be characterized by better industrial availability and lower toxicity compared to the cobalt-based systems, wherein direct further processing of the carbonylation products to carbonylation conversion products, for example conversion to polyurethanes, is also possible without prior removal of the catalyst system.

Surprisingly, it has been found that the object according to the invention is achieved by a process for the carbonylation of epoxides in the presence of catalyst systems, in which the carbonylation is carried out in the presence of carbon monoxide, characterized in that the catalyst system comprises a molybdenum-based compound.

### DETAILED DESCRIPTION

In one embodiment of the process according to the invention, the molybdenum-based compound is used in amounts of 0.0001 mol % to 20 mol %, based on the amount of epoxide.

In one embodiment of the process according to the invention, the molybdenum-based compound has an oxidation state of zero.

In one embodiment of the process according to the invention, the molybdenum-based compound is anionic.

In one embodiment of the process according to the invention, the molybdenum-based compound comprises one or more carbonyl ligands, preferably one to six, particularly preferably two to five.

In one embodiment of the process according to the invention, the molybdenum-based compound has a further ligand (L) other than the carbonyl ligand which, for mononuclear molybdenum-based compounds, typically results in the following functional relationship between the carbonyl ligand of the molybdenum-based compound having a total of 6 ligands and the ligand (L) of the molybdenum-based compound:

Mo(CO)₆₋ₓLₓ.

In one embodiment of the process according to the invention, the ligand (L) is one or more compound(s) and is selected from the group consisting of hydrido, such as (H), halide such as F, Cl, Br and I, pseudohalide such as CN, N₃, OCN, NCO, CNO, SCN, NCS and SeCN, inorganic N ligands such as NC, NO, NO₂, NO₃, NH₂, NCH₃, NCCH₃ and NCCF₃, pseudochalcogenides, carboxylates such as OTf, OAc and HCOO, other inorganic anions such as OH and HSO₄, allyl compounds such as η³-C₃H₅, dienes such as butadienes (C₄C₆), cyclic C₅ ligands such as cyclopentadienyl (Cp, η₅-C₅H₅) and pentamethylcyclopentadienyl (Cp*, η₅-C₅Me₅), alkyl compounds, aryl compounds, Fischer carbenes such as C(Ph)(Ph) (as Fischer carbene), C(OMe)(Ph) (as Fischer carbene), C(OEt)(NHPh) (as Fischer carbene), NHC carbenes such as 1,3-dimesitylimidazol-2-ylidene (IMes, NHC carbene) and 1,3-bis(2,4,6-trimethylphenyl)-4,5-dihydroimidazol-2-ylidene (SIMes, NHC carbene), amines such as NH₃, ethylenediamine, diethylenetriamine, aniline, pyridine, 2,2′-bipyridine and terpyridine, imines, aminophosphines such as PNP, PDI and PBP, phosphines such as PPh₃, PMe₃, PEt₃, PBu₃, PH₃, P(OMe)₃ and P(OEt)₃, phosphites, PEP pincer ligands where E=B and N, and ethers such as diethyl ether, THF and 2-Me-THF.

In a preferred embodiment of the process according to the invention, the ligand (L) is one or more compound(s) and is selected from the group consisting of H, F, Cl, Br, I, CN, NC, SCN, N₃, NO₂, NO₃, NH₂, OTf, OAc, OH, HSO₄, η³-C₃H₅, butadienes (C₄C₆), cyclopentadienyl (Cp, η⁵-C₅H₅), pentamethylcyclopentadienyl (Cp*, η⁵-C₅Me₅), C(Ph) (Ph) (as Fischer carbene), C(OMe)(Ph) (as Fischer carbene), C(OEt)(NHPh) (as Fischer carbene), 1,3-dimesitylimidazol-2-ylidene (IMes, NHC carbene), 1,3-bis(2,4,6-trimethylphenyl)-4,5-dihydroimidazol-2-ylidene (SIMes, NHC carbene), NH₃, ethylenediamine, diethylenetriamine, tetramethylethylenediamine, aniline, pyridine, 2,2′-bipyridine, PPh₃, PMe₃, PEt₃, PBu₃, PH₃, P(OMe)₃, P(OEt)₃, diethyl ether, THF and 2-Me-THF, preferably H, F, Cl, Br, I, CN, NC, SCN, N₃, NO₂, NO₃, NH₂, OTf, OAc, η³-C₃H₅, butadienes (C₄C₆), cyclopentadienyl (Cp, η⁵-C₅H₅), pentamethylcyclopentadienyl (Cp*, η⁵-C₅Me₅), C(Ph)(Ph) (as Fischer carbene), C(OMe)(Ph) (as Fischer carbene), C(OEt)(NHPh) (as Fischer carbene), 1,3-dimesitylimidazol-2-ylidene (IMes, NHC carbene), 1,3-bis(2,4,6-trimethylphenyl)-4,5-dihydroimidazol-2-ylidene (SIMes, NHC carbene), ethylenediamine, diethylenetriamine, tetramethylethylenediamine, aniline, pyridine, 2,2′-bipyridine, PPh₃ and PMe₃, particularly preferably H, F, Cl, Br, I, CN, NC, SCN, N₃, OTf, cyclopentadienyl (Cp, η⁵-C₅H₅), pentamethylcyclopentadienyl (Cp*, η⁵-C₅Me₅), C(Ph)(Ph) (as Fischer carbene), C(OMe)(Ph) (as Fischer carbene), C(OEt)(NHPh) (as Fischer carbene), 1,3-dimesitylimidazol-2-ylidene (IMes, NHC carbene) and 1,3-bis(2,4,6-trimethylphenyl)-4,5-dihydroimidazol-2-ylidene (SIMes, NHC carbene), especially preferably Cl, Br and pentamethylcyclopentadienyl (Cp*, η⁵-C₅Me₅).

In one embodiment of the process according to the invention, the catalyst system comprises an additional Lewis acid.

Lewis acids comprise one or more coordinatively unsaturated metal atoms as the Lewis acidic center, such as aluminum, tin, zinc, bismuth, vanadium, chromium, molybdenum, tungsten, iron, cobalt, nickel, rhodium, iridium, palladium, platinum, copper or zinc. However, the semi-metal boron also forms the Lewis acidic center of Lewis acids. Coordinatively unsaturated Lewis acidic centers are characterized by the fact that nucleophilic molecules can bind to them. Coordinatively unsaturated Lewis-acidic centers may already be present in the compound used as catalyst or forms in the reaction mixture, for example as a result of elimination of a weakly bonded nucleophilic molecule.

In one embodiment of the process according to the invention, Mo(V) chloride is used simultaneously as Lewis acid and precursor for generating a catalyst system according to the invention (cf. example 14 according to the invention). Under the reductive conditions, as prevail, for example, under typical reaction conditions according to the invention, such as pressure, CO-rich atmosphere, reaction temperatures up to 150° C., organic and non-protic dispersants, some Mo(V) can be reduced to Mo(0) and thereby forms the molybdenum component according to the invention of the catalyst system of the form [Mo(CO)ₙ(Cl)ₘ]^{m-}. The remaining fraction of Mo(V) chloride serves as the Lewis acid according to the invention. For the reduction of molybdenum chlorides in the presence of CO to prepare Mo(CO) complexes, reference is made to the disclosure in Georg Brauer (Ed.), inter alia: Handbuch der Präparativen Anorganischen Chemie [Handbook of Preparative Inorganic Chemistry], 3rd revised edition, Volume III, Ferdinand Enke, Stuttgart 1981, ISBN 3-432-87823-0, page 1634 ff. In a further embodiment of the process according to the invention, the synthesis of the catalyst systems can also take place in the reaction mixture itself and/or under reaction conditions. This approach is referred to as in situ generation of a catalyst system and was carried out in Example 15 according to the invention.

In one embodiment of the process according to the invention, the Lewis acid is cationic or charge-neutral, preferably cationic.

In a preferred embodiment of the process according to the invention, the Lewis acid is cationic.

In one embodiment of the process according to the invention, the cationic Lewis acid is an unsubstituted dicyclopentadienyl metal cation, a substituted dicyclopentadienyl metal cation, an unsubstituted metal porphyrin cation, a substituted metal porphyrin cation, an unsubstituted metal salen cation, a substituted metal salen cation, an unsubstituted metal salphen cation and/or a substituted metal salphen cation.

The Lewis acids may be simple inorganic, organometallic or organic compounds, such as BF₃, AlCl₃, FeCl₃, B(CH₃)₃, B(OH)₃, BPh₃, B(OR)₃, SiCl₄, SiF₄, PF₄, CO₂, SO₃ etc. and also adducts of these compounds. However, Lewis acids of more complex structure, in which the Lewis acidic center is coordinated by complex ligands, are also known and are preferably used in an embodiment of the process according to the invention. In particular, the ligands have the role of having a stabilising and/or reinforcing effect on the Lewis acid centers through partial coordinative saturation of the Lewis acid center(s). A distinction can be made between electronic and steric effects of the ligand molecules. Square-planar and square-tetrahedral coordination polyhedra between N and/or O ligand(s) and Lewis acidic centers are particularly suitable.

Particularly preferred as Lewis acids in one embodiment of the process according to the invention are cyclopentadienyl metal complexes, Lewis acids based on unsubstituted and substituted porphyrin, chlorin and corrin complexes, Lewis acids based on unsubstituted and substituted salen, salpn, salan, salalen, salph, salphen and salqu complexes, Lewis acids based on unsubstituted and substituted pincer complexes, Lewis acids based on unsubstituted and substituted pincer-diiminopyridine complexes.

Cyclopentadienyl metal complexes, which are complexes between one or more metals and one or more cyclopentadienyl derivative ligands (C₅R₅⁻), are characterized by the presence of so-called η⁵ metal-ligand bonds. In one embodiment of the process according to the invention, cyclopentadienyl metal complexes (Cp complexes), pentamethylcyclopentadienyl metal complexes (Cp* complexes), dicyclopentadienyl metal complexes ((Cp)2 complexes) and dipentamethylcyclopentadienyl metal complexes ((Cp*)2 complexes) are suitable as Lewis acids. In addition to these unsubstituted Cp rings and Cp* ring systems, all other conceivable substitution patterns are also possible. In one embodiment of the process according to the invention, the dicyclopentadienyl metal complex is [Cp₂Ti]⁺ and/or [Cp₂Ti(L)₂]⁺ where L=THF, Et₂O, PO and/or EO.

Lewis acids based on unsubstituted and substituted porphyrin, chlorin and corrin complexes with the Lewis acidic center selected from the groups of aluminum, tin, zinc, bismuth, vanadium, chromium, molybdenum, manganese, tungsten, iron, cobalt, nickel, rhodium, iridium, indium, cerium, lanthanum, yttrium, gadolinium, palladium, platinum, copper and zinc. All conceivable substitution patterns are possible. In one embodiment of the process according to the invention, the substituents are each independently selected from the group consisting of hydrogen (—H), methyl, tert-butyl, methoxy, phenyl, 4-methylphenyl, 4-methoxyphenyl, 4-chlorophenyl, 4-bromophenyl, 4-carboxylphenyl, 3,5-dimethoxyphenyl, 2-pyridyl, 4-pyridyl and N-methyl-4-pyridyl, preferably tert-butyl, methoxy, phenyl, 4-methylphenyl, 4-methoxyphenyl, 4-chlorophenyl or 3,5-dimethoxyphenyl, especially preferably phenyl.

Lewis acids based on unsubstituted and substituted salen, salpn, salan, salalen, salph, salphen and salqu complexes with the Lewis acidic center selected from the groups of aluminum, tin, zinc, bismuth, vanadium, chromium, molybdenum, manganese, tungsten, iron, cobalt, nickel, rhodium, iridium, indium, cerium, lanthanum, yttrium, gadolinium, palladium, platinum, copper and zinc. All conceivable substitution patterns are possible. In one embodiment of the process according to the invention, the substituents are each independently selected from the group consisting of hydrogen (—H), methyl, tert-butyl, phenyl, nitro, bromine, chlorine, hydroxyl, diethylamino and methoxy, preferably selected from the group of hydrogen (—H), methyl or tert-butyl, especially preferably tert-butyl.

Lewis acids based on unsubstituted and substituted pincer complexes with the Lewis acidic center selected from the groups of aluminum, tin, zinc, bismuth, vanadium, chromium, molybdenum, manganese, tungsten, iron, cobalt, nickel, ruthenium, rhodium, iridium, indium, cerium, lanthanum, yttrium, gadolinium, palladium, platinum, copper and zinc. In one embodiment of the process according to the invention, the ligands of these pincer complexes have the general form A-(organic linker)-E-(organic linker)-A, where A is selected from the group of the elements P, N and S and where E is selected is from the group of elements C, B and N, and are tridentate.

Lewis acids based on unsubstituted and substituted pincer-diiminopyridine complexes (abbreviation: DIP complexes) with the Lewis acidic center selected from the groups of aluminum, tin, zinc, bismuth, vanadium, chromium, molybdenum, manganese, tungsten, iron, cobalt, nickel, ruthenium, rhodium, iridium, indium, cerium, lanthanum, yttrium, gadolinium, palladium, platinum, copper and zinc. In one embodiment of the process according to the invention, the ligands of these DIP complexes are in the form of diiminopyridine and derivatives thereof.

In one embodiment of the process according to the invention, the catalyst system has the structure (I), (II), (III), (IV), (V), (VI) and/or (VII):

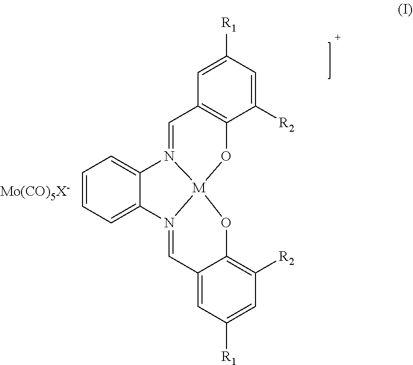

where X═H, F, Cl, Br, I, CN, NC, SCN, NCS, CP, N₃, NO₂, NO₃, NH₂, OTf, OH or HSO₄, preferably H, F, Cl, Br, I, CN, N₃ or OTf, especially preferably Cl or Br;
where M=Cr(III), Al(III), Fe(III), Co(III), Mn(III), V(III), In(III), Ga(III), Y(III), Ru(III), La(III), Ce(III), Gd(III) or Ir(III), preferably Cr(III), Al(III), Fe(III), Co(III), Mn(III) or Ga(III), especially preferably Cr(III) or Al(III);
where R₁ and R₂ are each independently selected from the group comprising hydrogen (—H), methyl, tert-butyl, phenyl, nitro, bromine, chlorine, hydroxyl, diethylamino and methoxy, preferably R₁ is identical to R₂, selected from the group of hydrogen (—H), methyl or tert-butyl, especially preferably tert-butyl;

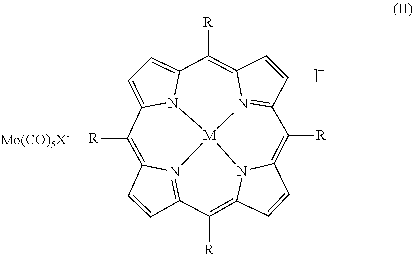

where X═H, F, Cl, Br, I, CN, NC, SCN, NCS, CP, N₃, NO₂, NO₃, NH₂, OTf, OH or HSO₄, preferably H, F, Cl, Br, I, CN, N₃ or OTf, especially preferably Cl or Br;
where M=Cr(III), Al(III), Fe(III), Co(III), Mn(III), V(III), In(III), Ga(III), Y(III), Ru(III), La(III), Ce(III), Gd(III) or Ir(III), preferably Cr(III), Al(III), Fe(III), Co(III), Mn(III) or Ga(III), especially preferably Cr(III) or Al(III);
where R is selected from the group comprising hydrogen (—H), methyl, tert-butyl, methoxy, phenyl, 4-methylphenyl, 4-methoxyphenyl, 4-chlorophenyl, 4-bromophenyl, 4-carboxylphenyl, 3,5-dimethoxyphenyl, 2-pyridyl, 4-pyridyl and N-methyl-4-pyridyl, preferably tert-butyl, methoxy, phenyl, 4-methylphenyl, 4-methoxyphenyl, 4-chlorophenyl or 3,5-dimethoxyphenyl, especially preferably phenyl.

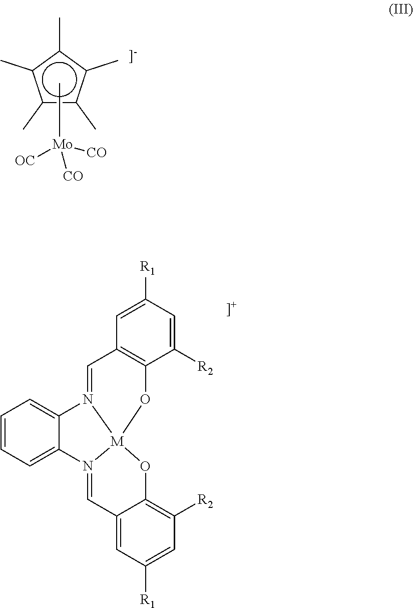

where M=Cr(III), Al(III), Fe(III), Co(III), Mn(III), V(III), In(III), Ga(III), Y(III), Ru(III), La(III), Ce(III), Gd(III) or Ir(III), preferably Cr(III), Al(III), Fe(III), Co(III), Mn(III) or Ga(III), especially preferably Cr(III) or Al(III);
where R₁ and R₂ are each independently selected from the group comprising hydrogen (—H), methyl, tert-butyl, phenyl, nitro, bromine, chlorine, hydroxyl, diethylamino and methoxy, preferably R₁ is identical to R₂, selected from the group of hydrogen (—H), methyl or tert-butyl, especially preferably tert-butyl;

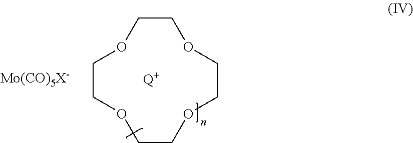

where X═H, F, Cl, Br, I, CN, NC, SCN, NCS, CP, N₃, NO₂, NO₃, NH₂, OTf, OH or HSO₄, preferably H, F, Cl, Br, I, CN, N₃ or OTf, especially preferably Cl or Br;
where Q=Li, Na, K, Rb, Cs, Cu or Ag, preferably Li, Na, K or Rb, especially preferably K;
where n=1-5, preferably 2-5, especially preferably 3;

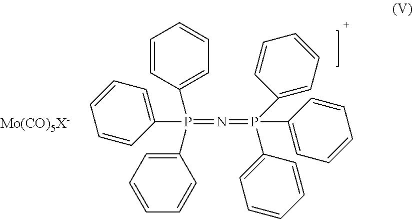

where X═H, F, Cl, Br, I, CN, NC, SCN, NCS, CP, N₃, NO₂, NO₃, NH₂, OTf, OH or HSO₄, preferably H, F, Cl, Br, I, CN, N₃ or OTf, especially preferably Cl or Br;

Q[Mo(CO)₅] (VI)
where Q=Li, Na, K, Rb, Cs, Cu or Ag, preferably Li, Na, K or Rb, especially preferably Na;
   and/or

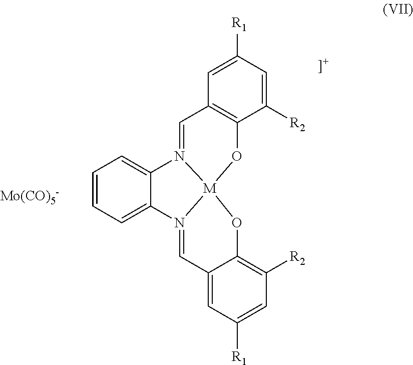

where M=Cr(III), Al(III), Fe(III), Co(III), Mn(III), V(III), In(III), Ga(III), Y(III), Ru(III), La(III), Ce(III), Gd(III) or Ir(III), preferably Cr(III), Al(III), Fe(III), Co(III), Mn(III) or Ga(III), especially preferably Cr(III) or Al(III);
where R₁ and R₂ are each independently selected from the group comprising hydrogen (—H), methyl, tert-butyl, phenyl, nitro, bromine, chlorine, hydroxyl, diethylamino and methoxy, preferably R₁ is identical to R₂, selected from the group of hydrogen (—H), methyl or tert-butyl, especially preferably tert-butyl.

In a preferred embodiment of the process according to the invention, the catalyst system has the structure (VIII), (IX), (X), (XI), (XII), (XIII), (XIV), (XV) and/or (XVI): (VIII)

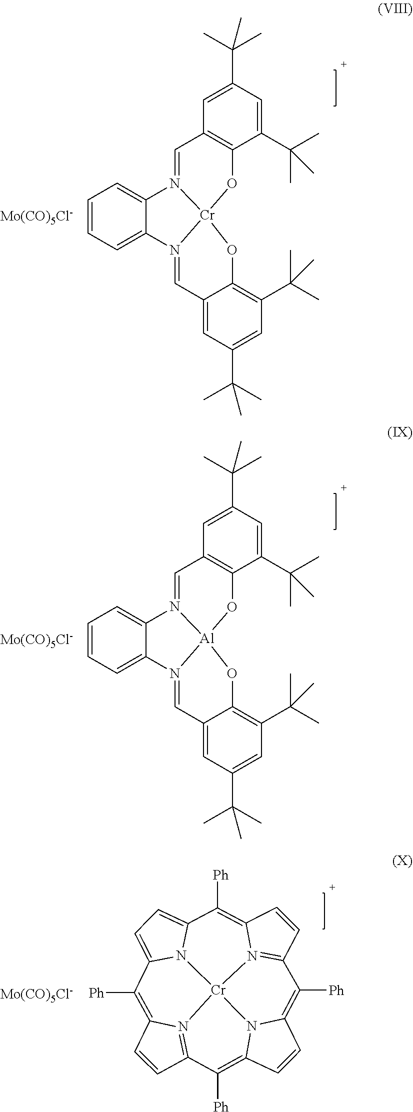

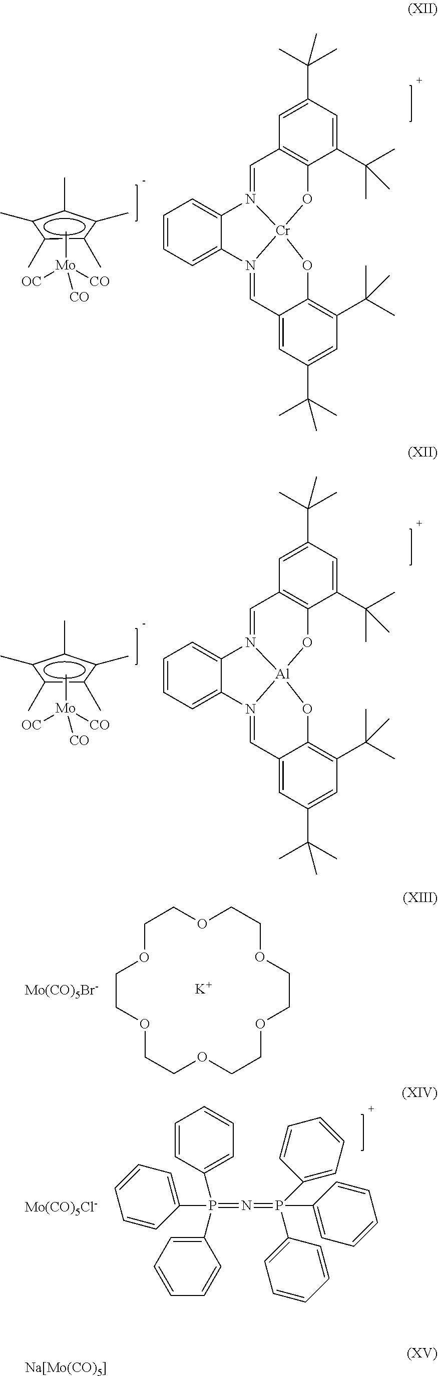

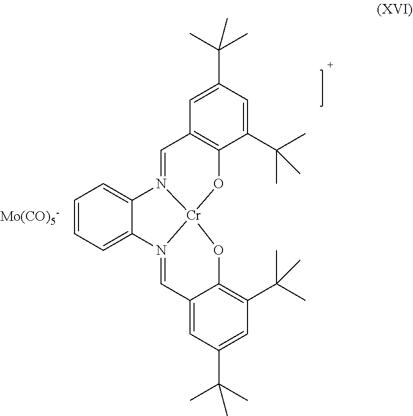

The epoxide according to the invention may be an epoxide having 2-45 carbon atoms. In a preferred embodiment of the process, the epoxide is selected from at least one compound from the group consisting of ethylene oxide, propylene oxide, 1-butene oxide, 2,3-butene oxide, 2-methyl-1,2-propene oxide (isobutene oxide), 1-pentene oxide, 2,3-pentene oxide, 2-methyl-1,2-butene oxide, 3-methyl-1,2-butene oxide, epoxides of C6-C22 α-olefins, such as 1-hexene oxide, 2,3-hexene oxide, 3,4-hexene oxide, 2-methyl-1,2-pentene oxide, 4-methyl-1,2-pentene oxide, 2-ethyl-1,2-butene oxide, 1-heptene oxide, 1-octene oxide, 1-nonene oxide, 1-decene oxide, 1-undecene oxide, 1-dodecene oxide, 4-methyl-1,2-pentene oxide, cyclopentene oxide, cyclohexene oxide, cycloheptene oxide, cyclooctene oxide, styrene oxide, methylstyrene oxide, pinene oxide, allyl glycedyl ether, vinylcyclohexene oxide, cyclooctadiene monoepoxide, cyclododecatriene monoepoxide, butadiene monoepoxide, isoprene monoepoxide, limonene oxide, 1,4-divinylbenzene monoepoxide, 1,3-divinylbenzene monoepoxide, glycidyl acrylate and glycidylmethacrylate, mono- or polyepoxidized fats as mono-, di- and triglycerides, epoxidized fatty acids, C1-C24 esters of epoxidized fatty acids, epichlorohydrin, glycidol, and derivatives of glycidol, for example glycidyl ethers of C1-C22 alkanols and glycidyl esters of C1-C22 alkanecarboxylic acids. Examples of derivatives of glycidol are phenyl glycidyl ether, cresyl glycidyl ether, methyl glycidyl ether, ethyl glycidyl ether and 2-ethylhexyl glycidyl ether.

In a particularly preferred embodiment of the process, the epoxide is ethylene oxide and/or propylene oxide.

In one embodiment of the process according to the invention, the carbonylation process is carried out in the presence of a suspension medium, preferably an aprotic suspension medium.

The suspension media used in accordance with the invention do not comprise any H-functional groups. Suitable suspension media are all polar aprotic, weakly polar aprotic and non-polar aprotic solvents, comprising no H-functional groups in each case. Suspension media used may also be a mixture of two or more of these suspension media. Mention is made by way of example at this point of the following polar aprotic solvents: 4-methyl-2-oxo-1,3-dioxolane (hereinafter also referred to as cyclic propylene carbonate or cPC), 1,3-dioxolan-2-one (hereinafter also referred to as cyclic ethylene carbonate or cEC), methyl formate, ethyl formate, isopropyl formate, propyl formate, ethyl acetate, isopropyl acetate, n-butyl acetate, methyl oxalate, 2,2-dimethoxypropane, acetone, methyl ethyl ketone, acetonitrile, benzonitrile, diethyl carbonate, dimethyl carbonate, nitromethane, dimethyl sulfoxide, sulfolane, dimethylformamide, dimethylacetamide and N-methylpyrrolidone. The group of non-polar and weakly polar aprotic solvents includes, for example, ethers such as dioxolane, dioxane, diethyl ether, methyl tert-butyl ether, ethyl tert-butyl ether, tert-amyl methyl ether, butyl methyl ether, methyl propyl ether, dimethyl ether, diisopropyl ether, ethyl methyl ether, methyl phenyl ether, dimethoxymethane, diethoxymethane, dimethoxyethane (glyme), [12] crown-4, cyclopentyl methyl ether, triglyme, tetraglyme, diethylene glycol dibutyl ether, 2-methyltetrahydrofuran and tetrahydrofuran, esters such as ethyl acetate and butyl acetate, hydrocarbons such as pentane, n-hexane, benzene and alkylated benzene derivatives (e.g. toluene, xylene, ethylbenzene) and chlorinated hydrocarbons such as chloroform, chlorobenzene, dichlorobenzene, fluorobenzene, difluorobenzene, methylene chloride and carbon tetrachloride. Preferred suspension media used are 4-methyl-2-oxo-1,3-dioxolane, 1,3-dioxolan-2-one, 2-methyltetrahydrofuran, tetrahydrofuran, dioxanes, dimethoxyethane (glyme), diethyl ether, ethyl acetate, methyl ethyl ketone, N-methylpyrrolidone, acetonitrile, sulfolane, dimethyl sulfoxide, dimethylformamide, toluene, xylene, ethylbenzene, dichlorobenzene, fluorobenzene, chlorobenzene and difluorobenzene and mixtures of two or more of these suspension media, particular preference being given to 4-methyl-2-oxo-1,3-dioxolane, 1,3-dioxolan-2-one, 2-methyltetrahydrofuran, tetrahydrofuran, dimethoxyethane (glyme), diethyl ether, ethyl acetate, acetonitrile, toluene, dichlorobenzene, fluorobenzene, chlorobenzene and difluorobenzene and mixtures of two or more of these suspension media.

In one embodiment of the process according to the invention, the process is carried out at a CO partial pressure of 60 to 130 bara.

In one embodiment of the process according to the invention, the process is carried out at temperatures of 0° C. to 200° C., preferably 60-190° C. and particularly preferably 80-180° C.

The present invention also relates to carbonylation products by reacting epoxides with carbon monoxide in accordance with the process according to the invention, in which the molar proportion of cyclic anhydrides, based on the epoxide used, is less than 5 mol %, wherein the proportion of cyclic anhydrides was determined by the ¹H-NMR method disclosed in the experimental section, with particular reference to Example 24.

Carbonylation products are understood to mean reaction products of epoxides with carbon monoxide, such as 4-ring lactones, beta-butyrolactone as reaction product of propylene oxide and CO, and propiolactone as reaction product of ethylene oxide and CO, and polyhydroxyalkanoates as polymerization product of 4-ring lactones, in particular polyhydroxybutyrate from beta-butyrolactone and polyhydroxypropionate from propiolactone, and also copolymers thereof which also have polyether repeating units in addition to the polyester repeating units mentioned. In accordance with the invention, carbonylation products also include reaction products of epoxides with carbon monoxide to give corresponding cyclic ester-ether compounds, in particular dioxepanone and dioxocanedione compounds, especially 1,4-dioxepan-5-one, 2,7-dimethyl-1,4-dioxepan-5-one, 2,6-dimethyl-1,4-dioxepan-5-one, 3,6-dimethyl-1,4-dioxepan-5-one, 3,7-dimethyl-1,4-dioxepan-5-one, 1, 5-dioxocane-2,6-dione, 3,8-dimethyl-1,5-dioxocane-2,6-dione, 3,7-dimethyl-1,5-dioxocane-2,6-dione and 4,8-dimethyl-1,5-dioxocane-2,6-dione.

Here, polyhydroxybutyrate (abbreviation: PHB)—

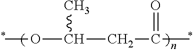

and/or
polyhydroxypropionate (abbreviation: PHP)

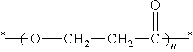

are present in the form of copolymers together with polyether repeating units,

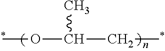

such as polyether repeating unit formed from PO and/or

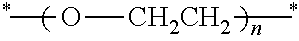

polyether repeating unit formed from EO (*=end groups and/or other repeating units).

The present invention further relates to a process for producing carbonylation conversion products, preferably polyurethanes, by reacting the carbonylation products according to the invention with epoxides, polyisocyanates and/or polycarboxylic acids, preferably with polyisocyanates.

The present invention also relates to the use of the molybdenum-based catalyst systems according to the invention for the carbonylation of epoxides.

In a first embodiment, the invention relates to a process for the carbonylation of epoxides in the presence of catalyst systems, characterized in that the catalyst system comprises a molybdenum-based compound.

In a second embodiment, the invention relates to a process according to the first embodiment, wherein the molybdenum-based compound comprises one or more carbonyl ligands, preferably one to six carbonyl ligands, particularly preferably two to five carbonyl ligands.

In a third embodiment, the invention relates to a process according to the first or second embodiment, wherein the molybdenum-based compound has a further ligand (L) other than the carbonyl ligand.

In a fourth embodiment, the invention relates to a process according to any of the first to third embodiments, wherein the ligand (L) is one or more compound (s) and is selected from the group consisting of H, F, Cl, Br, I, CN, NC, SCN, N₃, NO₂, NO₃, NH₂, OTf, OAc, OH, HSO₄, η³-C₃H₅, butadienes (C₄C₆), cyclopentadienyl (Cp, η⁵-C₅H₅), pentamethylcyclopentadienyl (Cp*, η⁵-C₅Me₅), C(Ph) (Ph) (as Fischer carbene), C(OMe)(Ph) (as Fischer carbene), C(OEt)(NHPh) (as Fischer carbene), 1,3-dimesitylimidazol-2-ylidene (IMes, NHC carbene), 1,3-bis(2,4,6-trimethylphenyl)-4,5-dihydroimidazol-2-ylidene (SIMes, NHC carbene), NH₃, ethylenediamine, diethylenetriamine, tetramethylethylenediamine, aniline, pyridine, 2,2′-bipyridine, PPh₃, PMe₃, PEt₃, PBu₃, PH₃, P(OMe)₃, P(OEt)₃, diethyl ether, THF and 2-Me-THF, preferably H, F, Cl, Br, I, CN, NC, SCN, N₃, NO₂, NO₃, NH₂, OTf, OAc, η³-C₃H₅, butadienes (C₄C₆), cyclopentadienyl (Cp, η⁵-C₅H₅), pentamethylcyclopentadienyl (Cp*, η⁵-C₅Me₅), C(Ph)(Ph) (as Fischer carbene), C(OMe)(Ph) (as Fischer carbene), C(OEt)(NHPh) (as Fischer carbene), 1,3-dimesitylimidazol-2-ylidene (IMes, NHC carbene), 1,3-bis(2,4,6-trimethylphenyl)-4,5-dihydroimidazol-2-ylidene (SIMes, NHC carbene), ethylenediamine, diethylenetriamine, tetramethylethylenediamine, aniline, pyridine, 2,2′-bipyridine, PPh₃ and PMe₃, particularly preferably H, F, Cl, Br, I, CN, NC, SCN, N₃, OTf, cyclopentadienyl (Cp, η⁵-C₅H₅), pentamethylcyclopentadienyl (Cp*, η⁵-C₅Me₅), C(Ph)(Ph) (as Fischer carbene), C(OMe)(Ph) (as Fischer carbene), C(OEt)(NHPh) (as Fischer carbene), 1,3-dimesitylimidazol-2-ylidene (IMes, NHC carbene) and 1,3-bis(2,4,6-trimethylphenyl)-4,5-dihydroimidazol-2-ylidene (SIMes, NHC carbene), especially preferably Cl, Br and pentamethylcyclopentadienyl (Cp*, η⁵-C₅Me₅).

In a fifth embodiment, the invention relates to a process according to any of the first to fourth embodiments, wherein the molybdenum in the molybdenum-based compound has an oxidation state of zero.

In a sixth embodiment, the invention relates to a process according to any of the first to fifth embodiments, wherein the catalyst system comprises an additional Lewis acid.

In a seventh embodiment, the invention relates to a process according to the sixth embodiment, wherein the Lewis acid is cationic or charge-neutral, preferably cationic.

In an eighth embodiment, the invention relates to a process according to the seventh embodiment, wherein the Lewis acid is cationic.

In a ninth embodiment, the invention relates to a process according to the eighth embodiment, wherein the cationic Lewis acid is an unsubstituted dicyclopentadienyl metal cation, a substituted dicyclopentadienyl metal cation, an unsubstituted metal porphyrin cation, a substituted metal porphyrin cation, an unsubstituted metal salen cation, a substituted metal salen cation, an unsubstituted metal salphen cation and/or a substituted metal salphen cation.

In a tenth embodiment, the invention relates to a process according to the eighth or ninth embodiment, wherein the molybdenum-based compound is anionic.

In an eleventh embodiment, the invention relates to a process according to any of the first to tenth embodiments, wherein the catalyst system has the structure (I), (II), (III), (IV), (V), (VI) and/or (VII):

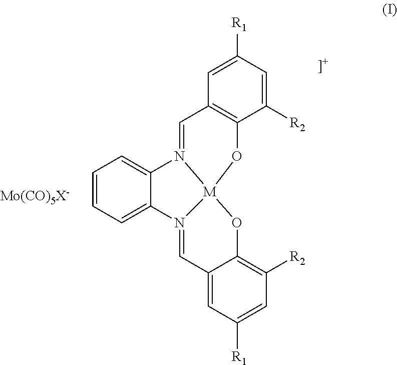

where X═H, F, Cl, Br, I, CN, NC, SCN, NCS, CP, N₃, NO₂, NO₃, NH₂, OTf, OH or HSO₄, preferably H, F, Cl, Br, I, CN, N₃ or OTf, especially preferably Cl or Br;
where M=Cr(III), Al(III), Fe(III), Co(III), Mn(III), V(III), In(III), Ga(III), Y(III), Ru(III), La(III), Ce(III), Gd(III) or Ir(III), preferably Cr(III), Al(III), Fe(III), Co(III), Mn(III) or Ga(III), especially preferably Cr(III) or Al(III);
where R₁ and R₂ are each independently selected from the group comprising hydrogen (—H), methyl, tert-butyl, phenyl, nitro, bromine, chlorine, hydroxyl, diethylamino and methoxy, preferably R₁ is identical to R₂, selected from the group of hydrogen (—H), methyl or tert-butyl, especially preferably tert-butyl; (II)

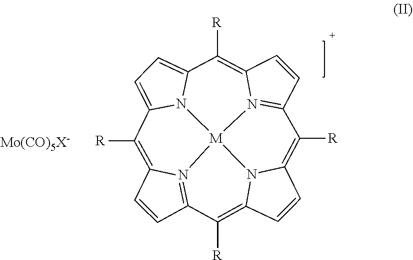

where X═H, F, Cl, Br, I, CN, NC, SCN, NCS, CP, N₃, NO₂, NO₃, NH₂, OTf, OH or HSO₄, preferably H, F, Cl, Br, I, CN, N₃ or OTf, especially preferably Cl or Br;
where M=Cr(III), Al(III), Fe(III), Co(III), Mn(III), V(III), In(III), Ga(III), Y(III), Ru(III), La(III), Ce(III), Gd(III) or Ir(III), preferably Cr(III), Al(III), Fe(III), Co(III), Mn(III) or Ga(III), especially preferably Cr(III) or Al(III);
where R is selected from the group comprising hydrogen (—H), methyl, tert-butyl, methoxy, phenyl, 4-methylphenyl, 4-methoxyphenyl, 4-chlorophenyl, 4-bromophenyl, 4-carboxylphenyl, 3,5-dimethoxyphenyl, 2-pyridyl, 4-pyridyl and N-methyl-4-pyridyl, preferably tert-butyl, methoxy, phenyl, 4-methylphenyl, 4-methoxyphenyl, 4-chlorophenyl or 3,5-dimethoxyphenyl, especially preferably phenyl.

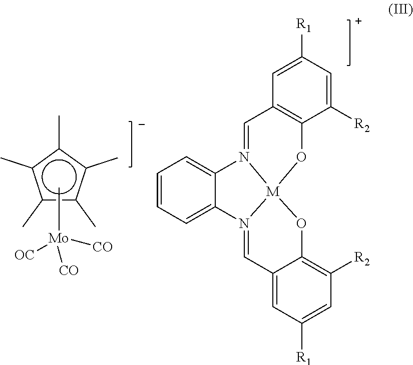

where M=Cr(III), Al(III), Fe(III), Co(III), Mn(III), V(III), In(III), Ga(III), Y(III), Ru(III), La(III), Ce(III), Gd(III) or Ir(III), preferably Cr(III), Al(III), Fe(III), Co(III), Mn(III) or Ga(III), especially preferably Cr(III) or Al(III);
where R₁ and R₂ are each independently selected from the group comprising hydrogen (—H), methyl, tert-butyl, phenyl, nitro, bromine, chlorine, hydroxyl, diethylamino and methoxy, preferably R₁ is identical to R₂, selected from the group of hydrogen (—H), methyl or tert-butyl, especially preferably tert-butyl;

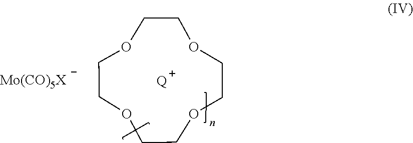

where X═H, F, Cl, Br, I, CN, NC, SCN, NCS, CP, N₃, NO₂, NO₃, NH₂, OTf, OH or HSO₄, preferably H, F, Cl, Br, I, CN, N₃ or OTf, especially preferably Cl or Br;
where Q=Li, Na, K, Rb, Cs, Cu or Ag, preferably Li, Na, K or Rb, especially preferably K;
where n=1-5, preferably 2-5, especially preferably 3;

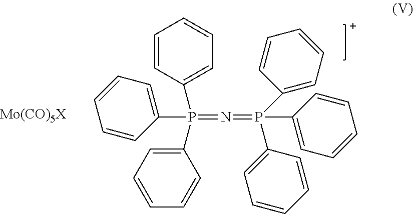

where X═H, F, Cl, Br, I, CN, NC, SCN, NCS, CP, N₃, NO₂, NO₃, NH₂, OTf, OH or HSO₄, preferably H, F, Cl, Br, I, CN, N₃ or OTf, especially preferably Cl or Br;

Q[Mo(CO)₅] (VI)
where Q=Li, Na, K, Rb, Cs, Cu or Ag, preferably Li, Na, K or Rb, especially preferably Na;
   and/or

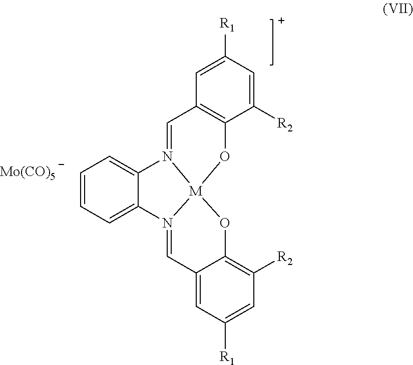

where M=Cr(III), Al(III), Fe(III), Co(III), Mn(III), V(III), In(III), Ga(III), Y(III), Ru(III), La(III), Ce(III), Gd(III) or Ir(III), preferably Cr(III), Al(III), Fe(III), Co(III), Mn(III) or Ga(III), especially preferably Cr(III) or Al(III);
where R₁ and R₂ are each independently selected from the group comprising hydrogen (—H), methyl, tert-butyl, phenyl, nitro, bromine, chlorine, hydroxyl, diethylamino and methoxy, preferably R₁ is identical to R₂, selected from the group of hydrogen (—H), methyl or tert-butyl, especially preferably tert-butyl.

In the twelfth embodiment, the invention relates to carbonylation products obtainable according to at least one of the first to eleventh embodiments, wherein the molar proportion of cyclic anhydrides, based on the epoxide used, is less than 5 mol %, wherein the proportion of cyclic was determined by the 1H-NMR method disclosed in the experimental section.

In a thirteenth embodiment, the invention relates to a process for the production of carbonylation conversion products, preferably polyurethanes, by reacting the carbonylation products according to the twelfth embodiment with epoxides, polyisocyanates and/or polycarboxylic acids, preferably with polyisocyanates.

In a fourteenth embodiment, the invention relates to the use of the catalyst systems comprising a molybdenum-based compound according to any of the first to eleventh embodiments for the carbonylation of epoxides.

In a fifteenth embodiment, the invention relates to a process according to any of the first to twelfth embodiments, wherein the catalyst system has the structure (VIII), (IX), (X), (XI), (XII), (XIII), (XIV), (XV) and/or (XVI):

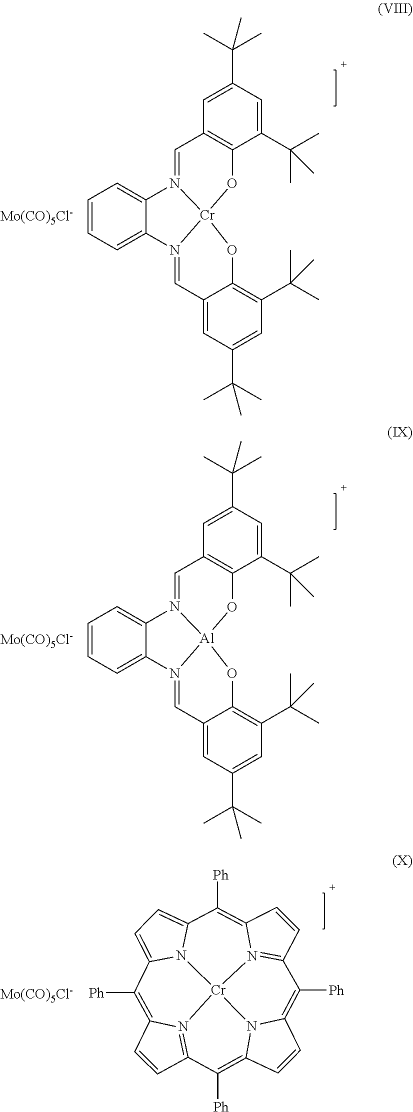

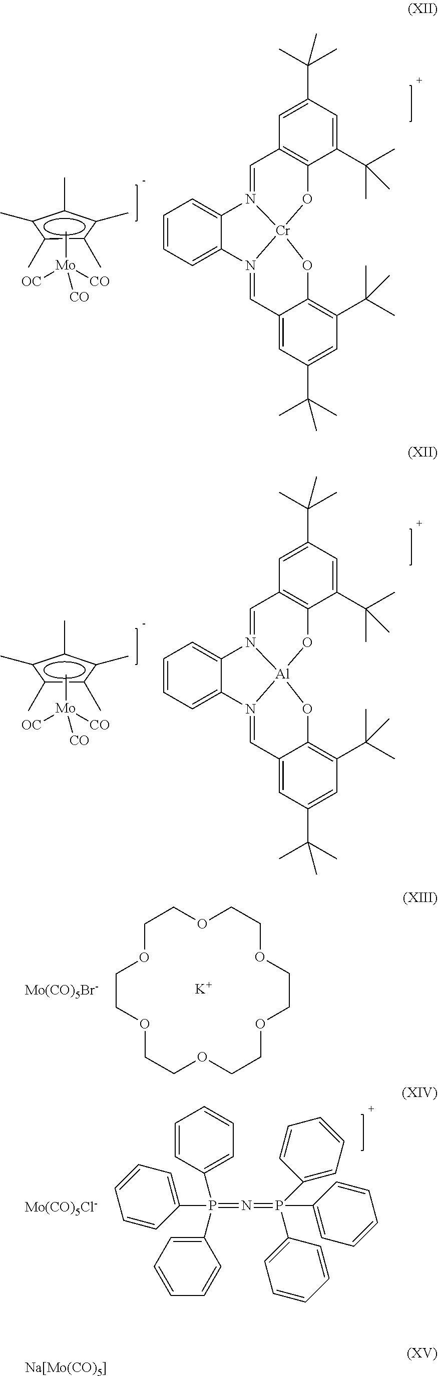

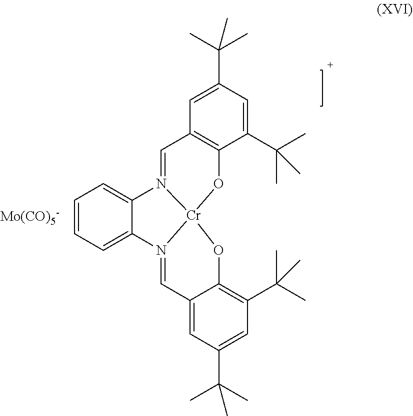

### EXAMPLES

Starting Materials Used:

Argon, abbreviation Ar, 99.998%, Westfalen AG

Deuterated chloroform, CDCl₃, 99.8%, Eurisotop

Deuterated tetrahydrofuran, THF-d₈, 99.5%, Eurisotop

ARCOL® POLYOL 1004, abbreviation: PET 1004, Covestro Deutschland AG

beta-Butyrolactone, abbreviation: bBL, 98%, Sigma Aldrich Chemie GmbH

Toluene, dry, 99.85%, Acros Organics

Methanol, dry, 99.8%, Acros Organics

Chloroform, dry, 99.8%, Sigma Aldrich Chemie GmbH

Diethyl ether, Et₂O, abbreviation: ether, dry, 99.5%, Acros Organics

Trifluoromethanesulfonic acid, abbreviation: TfOH, 98%, Alfa Aesar

Diisopropylethylamine, 99%, Sigma Aldrich Chemie GmbH

Silica gel 60, abbreviation: SiO₂, Sigma Aldrich Chemie GmbH

Dichloromethane, abbreviation: DCM, dry, 99.8%, Acros Organics

Propiolactone, abbreviation: PL, 98.5%, Ferak Berlin GmbH

1-Butanol, 99.8%, Sigma Aldrich Chemie GmbH

3,5-Di-tert-butylsalicyladehyde, Sigma Aldrich Chemie GmbH

Et₂AlC1 (25% in toluene), Sigma Aldrich Chemie GmbH

Chromium(II) chloride, CrCl₂, dry, 99.9%, Strem Chemicals, Inc.

Molybdenum hexacarbonyl, Mo (CO)₆, ≥99.9%, Sigma Aldrich Chemie GmbH

Tetrahydrofuran, abbreviation: THF, dry, 99.5%, Acros Organics

n-Hexane, abbreviation: Hexane, dry, 97%, Acros Organics

Tetraphenylporphyrinchromium(III) chloride, abbreviation: (TPP)CrCl, abcr GmbH

Cyclopentadienyllithium, abbreviation: Cp*Li, 97%, Sigma Aldrich Chemie GmbH

Celite® 545, abbreviation: Celite, Acros Organics

n-Pentane, abbreviation: pentane, dry, 99%, Acros Organics

[18] Crown-6, 99.0%, Sigma Aldrich Chemie GmbH

Potassium bromide, KBr, 99%, Acros Organics

Naphthalene, abbreviation: Nap, 99%, Sigma Aldrich Chemie GmbH

Sodium in mineral oil, abbreviation: Na, 99.8%, Acros Organics

Dicobalt octacarbonyl, Co₂(CO)₈, ≥90% (1-10% hexane), Sigma Aldrich Chemie GmbH

Bis(triphenylphosphoranylidene)ammonium chloride, 97%, Sigma Aldrich Chemie GmbH

Carbon monoxide, abbreviation: CO, 99.9%, Paxair Germany GmbH

Propylene oxide, abbreviation: PO, 99.5%, Sigma Aldrich Chemie GmbH

Ethylene oxide in THF, 2.5-3.3M, Sigma Aldrich Chemie GmbH

Dimethoxyethane, abbreviation: DME, dry, 99.5%, Acros Organics

1,4-Dioxane, abbreviation: dioxane, dry, 99.8%, Sigma Aldrich Chemie GmbH

Boron trifluoride etherate, BF₃-Et₂O, abbreviation: BF₃, Sigma Aldrich Chemie GmbH

Molecular sieve 3 Å, dried, Sigma Aldrich Chemie GmbH

Magnesium sulfate, MgSO₄, dry, Acros Organics

Sodium sulfate, Na₂SO₄, dry, Acros Organics

Sodium chloride, NaCl, dry, Acros Organics

Sodium hydroxide, NaOH, Honeywell

Dimethylformamide, abbreviation: DMF, Fischer Chemical

Molybdenum(V) chloride, 99.6%, Alfa Aesar

All liquids, with the exception of EO solutions, were degassed by multiple freeze-pump-thaw cycles and stored under an Ar protective gas atmosphere. Solids were degassed under vacuum and also stored under an Ar protective gas atmosphere. Propylene oxide was degassed by multiple freeze-pump-thaw cycles and stored at 0° C. under an Ar protective gas atmosphere and over 3 Å molecular sieves. Naphthalene was sublimed and stored in a glove box under vacuum.

IR Analysis:

All IR measurements were carried out on a Bruker Alpha-PFT-IR spectrometer. If necessary, the measurements were carried out under Ar protective gas. An automatic baseline correction was applied for all measured spectra. For further details on the IR analysis of the product mixtures, see “Quantitative IR analysis” below.

Nmr Analysis:

NMR spectra were recorded on a Bruker AV400 or AV300 spectrometer at room temperature. 1H-NMRs were measured at 400 or 300 MHz, 13C-NMRs correspondingly at 100 or 75 MHz. The 1H and 13C NMR signals are referenced to CHCl3 and TMS respectively.

Gc Analysis:

Gas chromatograms were recorded on a Thermo-Scientific instrument using a Trace-1 VF-WAX ms II capillary column (30 m×0.25 mm; film thickness=0.25 m), FID and He as carrier gas. Temperature profiles 40-220° C. at 8° C./min and 15 min iso at 220° C. Retention times (in min)=naphthalene (internal standard) 24.0, beta-butyrolactone 22.5, propylene oxide 5.1, ethylene oxide 4.4 and propiolactone 17.9.

Quantitative IR Analysis:

To calibrate the quantitative IR, mixtures of polyhydroxybutyrate and polyether with 0-25% by weight polyhydroxybutyrate fraction were prepared and measured. The calibration line results from plotting the relative absorbance against the relative fraction by weight of polyhydroxybutyrate. The relative absorbance is determined from the ratio of the absorbance of the C═O band to that of the C—O band. The relative proportion by mass (in wt %) of polyhydroxybutyrate repeating units to polyether repeating units—in non-volatile reaction residues (cf. general test procedure)—was therefore calculated as follows: w(polyhydroxybutyrate)=relative absorbance/0.0145. The procedure for polyhydroxypropionate was analogous. The relative proportion by mass (in wt %) of polyhydroxypropionate repeating units to polyether repeating units—in non-volatile reaction residues (cf. general test procedure)—was therefore calculated as follows: w(polyhydroxypropionate)=relative absorbance/0.053.

Polyether (PET 1004 for Short) as Reference Polymer:

ARCOL® POLYOL 1004 (PET 1004 for short) from Covestro was used as polyether reference polymer for quantitative IR and NMR analysis. This is a bifunctional polyether polyol based on PO having an average MW of ca. 435 g/mol.

IR: ν=1087 (C—O) cm⁻¹.

¹H-NMR (CDCl₃, 7.26 ppm): 1.07-1.16 (m, CH₃), 3.14-3.92 (m, CH, CH₂);

¹³C-NMR (CDCl₃, 77.16 ppm): 17.0-18.6, 65.7-67.3, 73.4-76.1 ppm

### Synthesis of Polyhydroxybutyrate (Abbreviation PHB)

Polyhydroxybutyrate (PHB for short) as reference polymer for quantitative IR and NMR analysis is synthesized in accordance with Tetrahedron Asymmetry, 2003, 14, 3249-3252 or Polym. Chem., 2014, 5, 161-168. Under an Ar protective gas atmosphere, 3.8 mL of β-butyrolactone and 40 mL of toluene were initially charged in a Schlenk flask, 0.07 mL each of methanol and trifluoromethanesulfonic acid were added and the mixture was then stirred at 30° C. for 2 hours. It was then quenched with 0.15 mL of diisopropylethylamine, concentrated on a rotary evaporator, purified by column chromatography (stationary phase: SiO₂, eluent: DCM/MeOH 20:1 v/v) and freed from solvent. The PHB reference polymer was finally dried in a high vacuum and analyzed by means of IR and NMR.

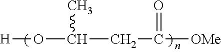

IR: ν=1731 (C═O) cm⁻¹.

¹H-NMR (CDCl₃, 7.26 ppm): 5.25 (s, CH), 3.67 (s, OCH₃) 2.4-2.63 (d, CH₂), 1.25-1.28 (d, CH₃) ppm.

¹³C-NMR (CDCl₃, 77.16 ppm): 20.0 (CH₃), 41.0 (CH₂), 67.8 (CH), 169.4 (C═O) ppm.

### Synthesis of Polyhydroxypropionate (Abbreviation: PHP)

The polyhydroxypropionate (PHP for short) as a reference polymer for quantitative IR and NMR analysis is synthesized analogously to PHB. Under an Ar protective gas atmosphere, 2.93 mL of propiolactone and 40 mL of toluene were initially charged in a Schlenk flask, 0.07 mL each of methanol and trifluoromethanesulfonic acid were added and the mixture was then stirred at room temperature for 0.5 hours. It was then quenched with 0.15 mL of diisopropylethylamine, filtered and washed with toluene and diethyl ether. The PHP reference polymer was finally dried in a high vacuum and analyzed by means of IR and NMR.

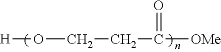

IR: ν=1725 (C═O) cm⁻¹.

¹H-NMR (CDCl₃, 7.26 ppm): 2.55 (t, CH₂), 4.22 (m, CH₂) ppm.

¹³C-NMR (CDCl₃, 77.16 ppm); 33.4 (CH₂), 59.9 (CH₂), 170.1 (C═O) ppm.

### Synthesis of the Salphen Complex Salt A

The salphen complex salt A was synthesized according to Jiang et al. in Top. Catal., 2017, 60, 750-754 and an isolated yield of 77% was obtained.

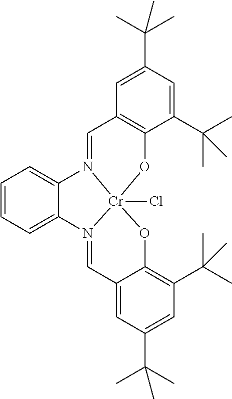

### Synthesis of the Salphen Complex Salt B

The salphen complex salt B was synthesized according to Getzler et al. in J. Am. Chem. Soc., 2002, 124, 1174-1175 and an isolated yield of 91% was obtained.

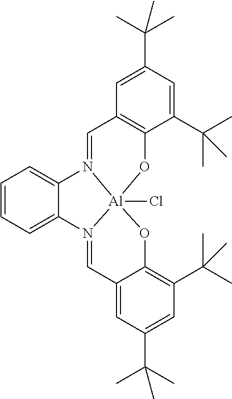

### Synthesis of Catalyst System 1 of the Formula (VIII)

Mo(CO)₆ (0.42 g, 1.60 mmol) and salphen complex salt A (1 g, 1.60 mmol) were initially charged under an Ar protective gas atmosphere in a Schlenk flask equipped with reflux condenser, 35 mL of THF were added and the mixture was then boiled under reflux for 5 hours. The mixture was cooled to room temperature, filtered and the resulting filtrate was concentrated fully to dryness under reduced pressure. The catalyst system 1 was obtained in 87% (1.4 g, 1.40 mmol) isolated yield and examined by IR. Characteristic carbonyl bands were identified at 2027, 1943 and 1864 cm⁻¹.

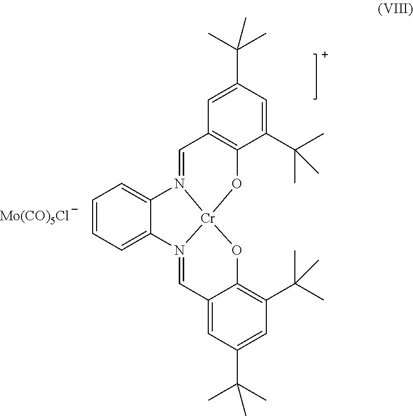

### Synthesis of Catalyst System 2 of the Formula (IX)

Mo(CO)₆ (0.44 g, 1.67 mmol) and salphen complex salt B (1 g, 1.67 mmol) were initially charged under an Ar protective gas atmosphere in a Schlenk flask equipped with reflux condenser, 50 mL of THF were added and the mixture was then boiled under reflux for 17 hours. This was cooled to room temperature, filtered, the resulting filtrate was concentrated fully to dryness under reduced pressure, washed with hexane and dried again. The catalyst system 2 was obtained in 36% (0.56 g, 0.36 mmol) isolated yield and examined by IR. Characteristic carbonyl bands were identified at 2071, 1922 and 1848 cm⁻¹.

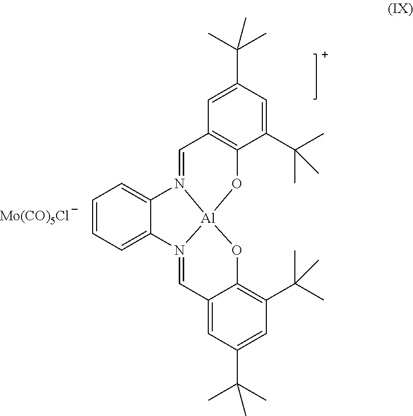

### Synthesis of Catalyst System 3 of the Formula (X)

Mo(CO)₆ (0.095 g, 0.36 mmol) and (TPP)CrCl (1 g, 1.67 mmol) were initially charged under an Ar protective gas atmosphere in a Schlenk flask wrapped in aluminum foil, equipped with a reflux condenser, 40 mL of THF were added and the mixture was boiled under reflux for 3 hours. This was cooled to room temperature, the resulting reaction mixture was concentrated fully to dryness under reduced pressure, washed twice with hexane and dried again. The catalyst system 3 was obtained in 53% (0.19 g, 0.17 mmol) isolated yield and examined by IR. Characteristic carbonyl bands were identified at 2060, 1932 and 1871 cm⁻¹.

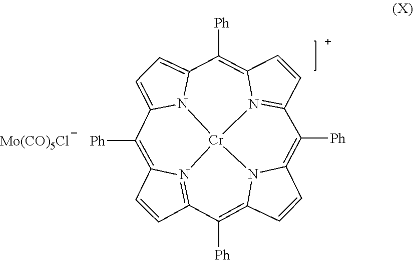

### Synthesis of Catalyst System 4 of the Formula (XI)

Mo(CO)₆ (1.80 g, 7.03 mmol) and Cp*Li (1 g, 7.03 mmol) were initially charged under an Ar protective gas atmosphere in a Schlenk flask equipped with reflux condenser, 35 mL of THF were added and the mixture was then boiled under reflux for 22 hours. This was cooled to room temperature, the resulting reaction mixture was concentrated fully to dryness under reduced pressure, washed twice with hexane and dried again. The slightly yellowish, solid intermediate Li[Cp*Mo(CO)₃] was obtained in 86% (1.95 g, 6.06 mmol) isolated yield and examined by IR (characteristic IR bands: ν=2060, 1932 and 1871 cm⁻¹). Subsequently, Li[Cp*Mo(CO)₃] (0.96 g, 2.97 mmol) and salphen complex salt A (1 g, 2.97 mmol) were initially charged under an Ar protective gas atmosphere in a Schlenk flask, 150 mL of THF were added and the mixture was stirred overnight. The resulting reaction mixture was filtered over celite, then concentrated fully to dryness under reduced pressure, washed twice with cold pentane and dried again. The catalyst system 4 was obtained in 61% (1.92 g, 1.80 mmol) isolated yield and examined by IR. Characteristic carbonyl bands were identified at 1921 and 1868 cm⁻¹.

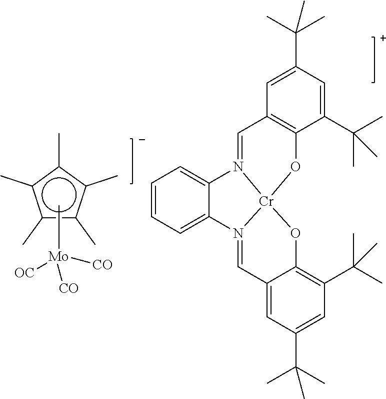

### Synthesis of Catalyst System 5 of the Formula (XII)

The intermediate Li[Cp*Mo(CO)₃] was prepared in analogous manner as for catalyst system 4. Subsequently, Li[Cp*Mo(CO)₃] (0.27 g, 0.83 mmol) and salphen complex salt B (0.5 g, 0.83 mmol) were initially charged under an Ar protective gas atmosphere in a Schlenk flask, 40 mL of THF were added and the mixture was stirred overnight. The resulting reaction mixture was filtered over celite and then concentrated fully to dryness under reduced pressure. The catalyst system 5 was obtained in 44% (0.38 g, 0.37 mmol) isolated yield and examined by IR. Characteristic carbonyl bands were identified at 1916 and 1865 cm⁻¹.

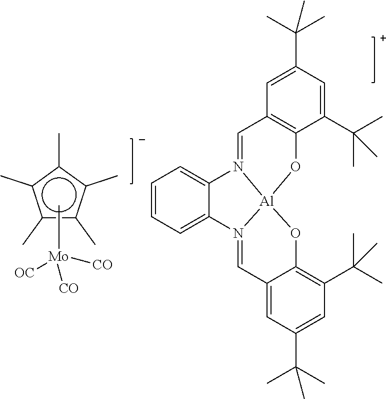

### Synthesis of Catalyst System 6 of the Formula (XIII)

The catalyst system 6 was synthesized according to WO 2007/073225 A1 and an isolated yield of 87% was obtained.

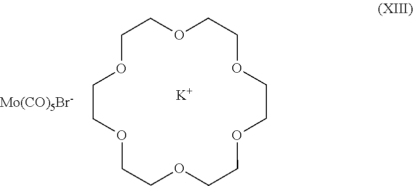 [SMILES: "Mo(CO)5Br-" ; C1COCCOCCOCCOCCOCCO1 ; [K+]]

### Synthesis of Catalyst System 7 of the Formula (XIV)

Mo(CO)₆ (0.20 g, 0.76 mmol) and (PPN)Cl (0.43 g, 0.76 mmol) were initially charged under an Ar protective gas atmosphere in a Schlenk flask equipped with reflux condenser, 5 mL of THF were added and the mixture was then boiled under reflux for 2 hours. This was cooled to room temperature, filtered and the resulting filtrate was cooled to 0° C., hexane was added and the mixture was stored at 0° C. for two days. The liquid phase was then decanted off and the residue washed with DCM and concentrated fully to dryness under reduced pressure. The catalyst system 7 was obtained in 54% (0.33 g, 0.41 mmol) isolated yield and examined by IR. Characteristic carbonyl bands were identified at 2065, 1900 and 1848 cm⁻¹.

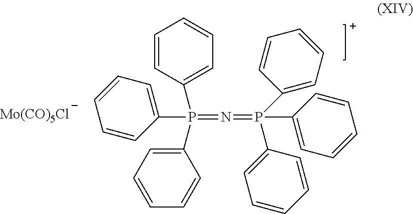

### Synthesis of Catalyst System 8 of the Formula (XV)

Freshly sublimed naphthalene (3.16 g, 24.60 mmol) and finely cut sodium (0.53 g, 23.40 mmol) were initially charged in a Schlenk flask under an Ar protective gas atmosphere, 55 ml of THF were added and the mixture was then stirred for two days. The green sodium naphthenate solution obtained was stored at −26° C. Then, Mo(CO)₆ (1 g, 3.80 mmol) and 10.5 mL of THF were initially charged in a Schlenk flask, cooled to −60° C. and the previously prepared sodium naphthenate solution was added dropwise with stirring. After the addition was complete, the mixture was slowly warmed to room temperature and stirred for a further 24 hours. The reaction mixture was then concentrated fully to dryness under reduced pressure, waxed three times with hexane and again concentrated fully to dryness under reduced pressure. The catalyst system 8 (formula: Na[Mo(CO)₅]) was obtained in 87% (0.86 g, 3.30 mmol) isolated yield and examined by IR. Characteristic carbonyl bands were identified at 1864 and 1733 cm⁻¹.

### Synthesis of Catalyst System 9 of the Formula (XVI)

Na[Mo(CO)₅] (catalyst system 8, 0.1 g, 0.38 mmol) and salphen complex salt A (0.24 g, 0.38 mmol) were initially charged in a Schlenk flask under an Ar protective gas atmosphere, 10 mL of THF were added and the mixture was then stirred for 22 hours. The resulting reaction mixture was filtered and the filtrate was concentrated fully to dryness under reduced pressure. The catalyst system 9 was obtained in 44% (0.38 g, 0.0.37 mmol) isolated yield and examined by IR. Characteristic carbonyl bands were identified at 1931 and 1864 cm⁻¹.

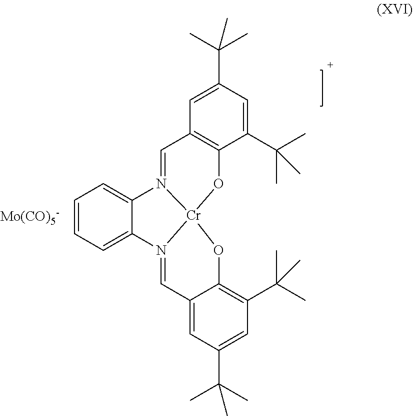

### Synthesis of Catalyst System 10 of the Formula (XVII) (Comparison)

The catalyst system 10 was synthesized according to Kramer et al. in Org. Lett., 2006, 8, 3709-3712 and an isolated yield of 68% was obtained.

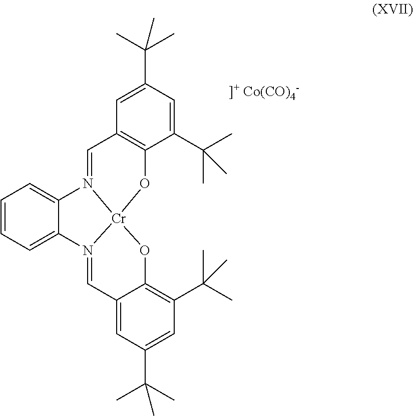

General Experimental Procedure:

The catalyst system, suspension medium, naphthalene as internal standard and epoxide were weighed into a Schlenk tube and stirred under a countercurrent of Ar protective gas. The total volume of the mixture was 2-5 mL. This mixture was then completely transferred to a 10 mL pressure reactor under a gentle CO gas countercurrent. The desired CO pre-pressure was adjusted while stirring, heated to reaction temperature and the initial pressure at the reaction temperature was determined. At the end of the reaction time, the final pressure at reaction temperature was determined and the pressure reactor was then cooled with the aid of a water/ice mixture. The remaining residual pressure was slowly released and a sample of the reaction mixture was immediately taken for analysis. After filtration through a short path column with celite as the stationary phase, part of the sample was analyzed by means of NMR and GC. Another part of the sample was freed from volatile constituents under reduced pressure and analyzed by quantitative IR and NMR. The polymer yield was determined gravimetrically, taking into account the remaining catalyst. The volatile constituents were collected with the aid of a cold trap, naphthalene was added as external standard and the mixture was also analyzed.

### Example 1: Carbonylation of Propylene Oxide Using Catalyst System 1

The experiment was carried out as described in the general experimental procedure. 1 mol % of catalyst system 1, THF as suspension medium, naphthalene as internal standard (0.009 mmol) and PO (0.924 mmol, 0.9M) were used. The reaction was carried out at 120° C., an initial pressure of 80 bar and a reaction time of 23 hours. A PO conversion of 85% was determined in the reaction mixture by NMR. By means of GC analysis of the reaction mixture, the formation of 3% beta-butyrolactone was detected. The non-volatile constituents were not isolated and not analyzed separately.

### Example 2: Carbonylation of Propylene Oxide Using Catalyst System 1

The experiment was carried out as described in the general experimental procedure. 1 mol % of catalyst system 1, THF as suspension medium, naphthalene as internal standard (1 mmol) and PO (10 mmol, 1.8M) were used. The reaction was carried out at 120° C., an initial pressure of 130 bar and a reaction time of 48 hours. A conversion of CO was observed, the difference between the initial and final pressure being 20 bar. The non-volatile constituents of the reaction mixture were isolated and analyzed. A polymer yield of 14% was determined with a proportion of 17% by weight PHB in the polymeric product.

### Example 3: Carbonylation of Propylene Oxide Using Catalyst System 1

The experiment was carried out as described in the general experimental procedure. 2 mol % of catalyst system 1, THF as suspension medium, naphthalene as internal standard (0.09 mmol) and PO (0.924 mmol, 0.9M) were used. The reaction was carried out at 100° C., an initial pressure of 60 bar and a reaction time of 46 hours. A PO conversion of 65% was determined in the reaction mixture by NMR. The non-volatile constituents of the reaction mixture were isolated and analyzed. A content of 17% by weight PHB in the polymeric product was determined.

### Example 4: Carbonylation of Propylene Oxide Using Catalyst System 1

The experiment was carried out as described in the general experimental procedure. 2 mol % of catalyst system 1, THF as suspension medium, naphthalene as internal standard (0.09 mmol) and PO (0.924 mmol, 0.9M) were used. The reaction was carried out at 150° C., an initial pressure of 100 bar and a reaction time of 23 hours. A PO conversion of 94% was determined in the reaction mixture by NMR. The non-volatile constituents of the reaction mixture were isolated and analyzed. A polymer yield of 50% was determined with a proportion of 45% by weight PHB in the polymeric product.

### Example 5: Carbonylation of Ethylene Oxide Using Catalyst System 1

The experiment was carried out as described in the general experimental procedure. 1 mol % of catalyst system 1, THF as suspension medium, naphthalene as internal standard (0.9 mmol) and EO (9.92 mmol, 2M) were used. The reaction was carried out at 150° C., an initial pressure of 100 bar and a reaction time of 87 hours. An EO conversion of 98% was determined in the reaction mixture by NMR. The non-volatile constituents of the reaction mixture were isolated and analyzed. A polymer yield of >99% with a proportion of 3% by weight PHP in the polymeric product was determined. The mass balance based on EO was >99%.

### Example 6: Carbonylation of Propylene Oxide Using Catalyst System 1

The experiment was carried out as described in the general experimental procedure. 1 mol % of catalyst system 1, DME as suspension medium, naphthalene as internal standard (0.2 mmol) and PO (2 mmol, 0.9M) were used. The reaction was carried out at 120° C., an initial pressure of 80 bar and a reaction time of 19 hours. The non-volatile constituents of the reaction mixture were isolated and analyzed. A proportion of 39% by weight PHB in the polymeric product was determined.

### Example 7: Carbonylation of Propylene Oxide Using Catalyst System 1

The experiment was carried out as described in the general experimental procedure. 2 mol % of catalyst system 1, DME as suspension medium, naphthalene as internal standard (0.2 mmol) and PO (2 mmol, 0.9M) were used. The reaction was carried out at 120° C., an initial pressure of 80 bar and a reaction time of 19 hours. A PO conversion of 69% was determined in the reaction mixture by NMR. The non-volatile constituents of the reaction mixture were isolated and analyzed. A polymer yield of 7% was determined with a proportion of 14% by weight PHB in the polymeric product.

### Example 8: Carbonylation of Propylene Oxide Using Catalyst System 1

The experiment was carried out as described in the general experimental procedure. 0.1 mol % of catalyst system 1, DME as suspension medium, naphthalene as internal standard (1 mmol) and PO (10 mmol, 1.8M) were used. The reaction was carried out at 150° C., an initial pressure of 70 bar and a reaction time of 65 hours. A conversion of CO was observed, the difference between the initial and final pressure being 13 bar. A PO conversion of 41% and the formation of <1% acetone were determined in the reaction mixture by NMR. The non-volatile constituents of the reaction mixture were isolated and analyzed. A polymer yield of 8% was determined with a proportion of 58% by weight PHB in the polymeric product.

### Example 9: Carbonylation of Propylene Oxide Using Catalyst System 1

The experiment was carried out as described in the general experimental procedure. 1 mol % of catalyst system 1, DME as suspension medium, naphthalene as internal standard (1 mmol) and PO (10 mmol, 1.8M) were used. The reaction was carried out at 150° C., an initial pressure of 70 bar and a reaction time of 65 hours. A conversion of CO was observed, the difference between the initial and final pressure being 15 bar. A PO conversion of 67% and the formation of 1.5% acetone were determined in the reaction mixture by NMR. The non-volatile constituents of the reaction mixture were isolated and analyzed. A polymer yield of 45% was determined with a proportion of 28% by weight PHB in the polymeric product.

### Example 10: Carbonylation of Ethylene Oxide Using Catalyst System 1

The experiment was carried out as described in the general experimental procedure. 1 mol % of catalyst system 1, a 2:1 mixture (v/v) of THF and DME as suspension medium, naphthalene as internal standard (0.9 mmol) and EO (9.92 mmol, 2M) were used. The reaction was carried out at 120° C., an initial pressure of 70 bar and a reaction time of 65 hours. An EO conversion of 97% was determined in the reaction mixture by NMR. The non-volatile constituents of the reaction mixture were isolated and analyzed. A polymer yield of >99% with a proportion of 2% by weight PHP in the polymeric product was determined. The mass balance based on EO was >99%.

### Example 11: Carbonylation of Propylene Oxide Using Catalyst System 1

The experiment was carried out as described in the general experimental procedure. 0.4 mol % of catalyst system 1, no suspending agent, naphthalene as internal standard (1 mmol) and PO (10 mmol) were used. The reaction was carried out at 120° C., an initial pressure of 80 bar and a reaction time of 19 hours. A PO conversion of 89% was determined in the reaction mixture by NMR. The non-volatile constituents of the reaction mixture were isolated and analyzed. A polymer yield of >8% with a proportion of 4% by weight PHB in the polymeric product was determined.

### Example 12: Carbonylation of Propylene Oxide Using Catalyst System 2

The experiment was carried out as described in the general experimental procedure. 1 mol % of catalyst system 2, DME, naphthalene as internal standard (1 mmol) and PO (10 mmol, 1.8 M) were used. The reaction was carried out at 150° C., an initial pressure of 70 bar and a reaction time of 65 hours. A PO conversion of 54% and the formation of 2.5% acetone were determined in the reaction mixture by NMR. The non-volatile constituents of the reaction mixture were isolated and analyzed. A polymer yield of >8% with a proportion of 16% by weight PHB in the polymeric product was determined.

### Example 13: Carbonylation of Propylene Oxide Using Catalyst System 3

The experiment was carried out as described in the general experimental procedure. 1 mol % of catalyst system 3, THF, naphthalene as internal standard (0.4 mmol) and PO (4 mmol, 0.9 M) were used. The reaction was carried out at 150° C., an initial pressure of 75 bar and a reaction time of 21 hours. A PO conversion of 78% and the formation of 2.3% acetone were determined in the reaction mixture by NMR. The non-volatile constituents of the reaction mixture were isolated and analyzed. A polymer yield of 48% was determined with a proportion of 14% by weight PHB in the polymeric product.

### Example 14: Carbonylation of Propylene Oxide Using Molybdenum(V) Chloride for In Situ Generation of a Catalyst System

The experiment was carried out as described in the general experimental procedure. 5 mol % molybdenum(V) chloride, DME, naphthalene as internal standard (1 mmol) and PO (10 mmol, 1.8M) were used. The reaction was carried out at 150° C., an initial pressure of 130 bar and a reaction time of 68 hours. A PO conversion of 100% was determined in the reaction mixture by NMR. The non-volatile constituents of the reaction mixture were isolated and analyzed. A polymer yield of 46% was determined with a proportion of 20% by weight PHB in the polymeric product.

### Example 15: Carbonylation of Propylene Oxide Using Molybdenum Hexacarbonyl and Salphen Complex Salt a for In Situ Generation of a Catalyst System

The experiment was carried out as described in the general experimental procedure. 1 mol % each of the two air-stable compounds molybdenum hexacarbonyl and salphen complex salt A, DME, naphthalene as internal standard (0.4 mmol) and PO (4 mmol, 1.8 M) were used. The reaction was carried out at 150° C., an initial pressure of 93 bar and a reaction time of 19 hours. A PO conversion of 30% was determined in the reaction mixture by NMR. The non-volatile constituents of the reaction mixture were isolated and analyzed. A proportion of 4% by weight PHB in the polymeric product was determined.

### Example 16: Carbonylation of Propylene Oxide Using Catalyst System 4

The experiment was carried out as described in the general experimental procedure. 3 mol % of catalyst system 4, a 1:7 mixture (v/v) of THF and 1,4-dioxane as suspension medium, naphthalene as internal standard (0.2 mmol) and PO (2 mmol, 0.5M) were used. The reaction was carried out at 150° C., an initial pressure of 124 bar and a reaction time of 63 hours. A PO conversion of 73% and a proportion of 8% acetone were determined in the reaction mixture by NMR. The non-volatile constituents of the reaction mixture were isolated and analyzed. A polymer yield of 61% was determined with a proportion of 70% by weight PHB in the polymeric product. The mass balance based on PO was 96%.

### Example 17: Carbonylation of Propylene Oxide Using Catalyst System 5

The experiment was carried out as described in the general experimental procedure. 1 mol % of catalyst system 5, THF, naphthalene as internal standard (0.4 mmol) and PO (4 mmol, 0.9 M) were used. The reaction was carried out at 150° C., an initial pressure of 103 bar and a reaction time of 63 hours. A PO conversion of 30% was determined in the reaction mixture by NMR. The non-volatile constituents of the reaction mixture were isolated and analyzed. A polymer yield of 13% was determined with a proportion of 25% by weight PHB in the polymeric product.

### Example 18 (Counter-Example) Carbonylation of Propylene Oxide without the Use of a Catalyst System

The experiment was carried out as described in the general experimental procedure, but without the addition of a catalyst system. DME, naphthalene as internal standard (1 mmol) and PO (10 mmol, 1.8M) were used. The reaction was carried out at 150° C., an initial pressure of 70 bar and a reaction time of 5 hours. A PO conversion of 2% was determined in the reaction mixture by NMR. Only traces of polymer could be found, but they did not comprise any PHB.

### Example 19: Carbonylation of Propylene Oxide Using Molybdenum Hexacarbonyl

The experiment was carried out as described in the general experimental procedure. 5 mol % molybdenum hexacarbonyl, THF, naphthalene as internal standard (1 mmol) and PO (10 mmol, 1.7M) were used. The reaction was carried out at 80° C., an initial pressure of 62 bar and a reaction time of 24 hours. A PO conversion of 11% was determined in the reaction mixture by NMR. Only traces of polymer could be found, but they did not comprise any PHB.

### Example 20: Carbonylation of Propylene Oxide Using Molybdenum Hexacarbonyl

The experiment was carried out as described in the general experimental procedure. 5 mol % molybdenum hexacarbonyl, DME, naphthalene as internal standard (1 mmol) and PO (10 mmol, 3.7M) were used. The reaction was carried out at 150° C., an initial pressure of 70 bar and a reaction time of 67 hours. A PO conversion of 50% was determined in the reaction mixture by NMR. Only traces of polymer could be found, but they did not comprise any PHB.

### Example 21: Carbonylation of Propylene Oxide Using Molybdenum Hexacarbonyl and Boron Trifluoride

The experiment was carried out as described in the general experimental procedure. 5 mol % molybdenum hexacarbonyl, 5 mol % boron trifluoride as the etherate, THF, naphthalene as internal standard (1 mmol) and PO (10 mmol, 1.7M) were used. The reaction was carried out at 80° C., an initial pressure of 62 bar and a reaction time of 24 hours. A PO conversion of 100% and the formation of 1% propanal and 3% 2-methyl-2-pentenal were determined in the reaction mixture by NMR. The non-volatile constituents of the reaction mixture were isolated and analyzed. A polymer yield of 84% was determined with a proportion of 3% by weight PHB in the polymeric product.

### Example 22: Carbonylation of Propylene Oxide Using Catalyst System 7

The experiment was carried out as described in the general experimental procedure. 5 mol % of catalyst system 7, THF, naphthalene as internal standard (0.1 mmol) and PO (1 mmol, 0.9M) were used. The reaction was carried out at 120° C., an initial pressure of 80 bar and a reaction time of 46 hours. A PO conversion of 100% was determined in the reaction mixture by NMR. Only traces of PHB could be detected.

### Example 23: Carbonylation of Propylene Oxide Using Catalyst System 8 and Boron Trifluoride

The experiment was carried out as described in the general experimental procedure. 2 mol % of catalyst system 8, 2.4 mol % boron trifluoride as the etherate, DME, naphthalene as internal standard (0.2 mmol) and PO (2 mmol, 0.9M) were used. The reaction was carried out at 120° C., an initial pressure of 75 bar and a reaction time of 45 hours. A PO conversion of 72% was determined in the reaction mixture by NMR. Only traces of PHB could be detected.

### Example 24 (Comparative): Carbonylation of Propylene Oxide Using Catalyst System 10

The experiment was carried out as described in the general experimental procedure. 1 mol % of catalyst system 10, THF, naphthalene as internal standard (0.2 mmol) and PO (2 mmol, 0.9M) were used. The reaction was carried out at 120° C., an initial pressure of 82 bar and a reaction time of 15 hours. A PO conversion of 100%, the formation of 77% methylsuccinic anhydride and 2% acetone could be detected in the reaction mixture by NMR. Only traces of polymer and no PHB could be detected.

The characterization and quantification of methylsuccinic anhydride is based on the specific ¹H-NMR signals and their integrals in relation to the PO/internal standard used:

¹H-NMR (CDCl₃, 7.26 ppm): 1.42 (d, CH₃), 2.57-2.67 (m, CH), 3.11-3.23 (m, CH₂);

¹³C-NMR (CDCl₃, 77.16 ppm): 16.1, 35.7, 36.1, 170.1, 174.6

TABLE 1
Working examples for the carbonylation of epoxides

| | | | | | | | Epoxide | Polymer | Proportion of |
|---|---|---|---|---|---|---|---|---|---|
| Ex. | Catalyst System | | Suspension | T | p | t | conversion | yield | PHB in the |
| No. | ([mol %]) | Epoxide | medium | [° C.] | [bar] | [h] | [%] | [%] | polymer [wt %] |
| 1 | 1 (1) | PO | THF | 120 | 80 | 23 | 85^{a} | n.d. | n.d. |
| 2 | 1 (1) | PO | THF | 120 | 130 | 48 | n.d. | 14 | 17 |
| 3 | 1 (2) | PO | THF | 100 | 60 | 46 | 65 | n.d. | 17 |
| 4 | 1 (2) | PO | THF | 150 | 100 | 23 | 94 | 50 | 45 |
| 5 | 1 (1) | EO | THF | 150 | 100 | 87 | 98 | 99 | 3^{b} |
| 6 | 1 (1) | PO | DME | 120 | 80 | 19 | n.d. | n.d. | 39 |
| 7 | 1 (2) | PO | DME | 120 | 80 | 19 | 69 | 7 | 14 |
| 8 | 1 (0.1) | PO | DME | 150 | 70 | 65 | 41 | 8 | 58 |
| 9 | 1 (1) | PO | DME | 150 | 70 | 65 | 67 | 45 | 28 |
| 10 | 1 (1) | EO | THF/DME | 120 | 70 | 65 | 97 | 99 | 2^{b} |
| | | | (2:1 v/v) | | | | | | |
| 11 | 1 (0.4) | PO | — | 120 | 80 | 19 | 89 | 8 | 4 |
| 12 | 2 (1) | PO | DME | 150 | 70 | 65 | 54 | 8 | 16 |
| 13 | 3 (1) | PO | THF | 150 | 75 | 21 | 78 | 48 | 14 |
| 14 | Molybdenum(V) | PO | DME | 150 | 130 | 68 | 100 | 46 | 20 |
| | chloride^{c} (5) | | | | | | | | |
| 15 | Molybdenum | PO | DME | 150 | 93 | 19 | 30 | n.d. | 4 |
| | hexacarbonyl (1) and | | | | | | | | |
| | salphen complex | | | | | | | | |
| | salt A^{c} (1) | | | | | | | | |
| 16 | 4 (3) | PO | THF/1,4-dioxane | 150 | 124 | 63 | 73 | 61 | 70 |
| | | | (1:7 v/v) | | | | | | |
| 17 | 5 (1) | PO | THF | 150 | 103 | 63 | 30 | 13 | 25 |
| 18 | — | PO | DME | 150 | 70 | 5 | 2 | Traces | 0 |
| 19 | Molybdenum | PO | THF | 80 | 62 | 24 | 11 | Traces | 0 |
| | hexacarbonyl (5) | | | | | | | | |
| 20 | Molybdenum | PO | DME | 150 | 70 | 67 | 50 | Traces | 0 |
| | hexacarbonyl (5) | | | | | | | | |
| 21 | Molybdenum | PO | THF | 80 | 62 | 24 | 100 | 84 | 3 |
| | hexacarbonyl (5) | | | | | | | | |
| | and boron | | | | | | | | |
| | trifluoride (5) | | | | | | | | |
| 22 | 7 (5) | PO | THF | 120 | 80 | 46 | 100 | n.d. | Traces |
| 23 | 8 (2) and boron | PO | DME | 120 | 75 | 45 | 72 | n.d. | Traces |
| | trifluoride (2,4) | | | | | | | | |
| 24 | 10 (1) | PO | THF | 120 | 82 | 15 | 100^{d} | Traces | 0 |
| (comp.) | | | | | | | | | |

n.d. = not determined
^{a}By means of GC analysis of the reaction mixture, the formation of 3% beta-butyrolactone was detected.
^{b}Figures for the proportion of PHP in the polymer in % by weight.
^{c}Precursor for in situ formation of an active catalyst system.
^{d}By means of ¹H— and ¹³C-NMR analysis of the reaction mixture, the formation of methylsuccinic anhydride in a yield of 77% could be detected

1. A process for the carbonylation of epoxides in the presence of catalyst systems, wherein the carbonylation is carried out in the presence of carbon monoxide, and wherein the catalyst system comprises a molybdenum-based compound.

2. The process as claimed in claim 1, wherein the molybdenum-based compound comprises one or more carbonyl ligands.

3. The process as claimed in claim 1, wherein the molybdenum-based compound has a further ligand (L) other than the carbonyl ligand.

4. The process as claimed in claim 3, wherein the ligand (L) is one or more compounds selected from the group consisting of H, F, Cl, Br, I, CN, NC, SCN, N₃, NO₂, NO₃, NH₂, OTf, OAc, OH, HSO₄, η³-C₃H₅, butadienes (C₄C₆), cyclopentadienyl (Cp, η⁵-C₅H₅), pentamethylcyclopentadienyl (Cp*, η⁵-C₅Me₅), C(Ph) (Ph) (as Fischer carbene), C(OMe)(Ph) (as Fischer carbene), C(OEt)(NHPh) (as Fischer carbene), 1,3-dimesitylimidazol-2-ylidene (IMes, NHC carbene), 1,3-bis(2,4,6-trimethylphenyl)-4,5-dihydroimidazol-2-ylidene (SIMes, NHC carbene), NH₃, ethylenediamine, diethylenetriamine, tetramethylethylenediamine, aniline, pyridine, 2,2′-bipyridine, PPh₃, PMe₃, PEt₃, PBu₃, PH₃, P(OMe)₃, P(OEt)₃, diethyl ether, THF and 2-Me-THF, preferably H, F, Cl, Br, I, CN, NC, SCN, N₃, NO₂, NO₃, NH₂, OTf, OAc, η³-C₃H₅, butadienes (C₄C₆), cyclopentadienyl (Cp, η⁵-C₅H₅), pentamethylcyclopentadienyl (Cp*, η⁵-C₅Me₅), C(Ph)(Ph) (as Fischer carbene), C(OMe)(Ph) (as Fischer carbene), C(OEt)(NHPh) (as Fischer carbene), 1,3-dimesitylimidazol-2-ylidene (IMes, NHC carbene), 1,3-bis(2,4,6-trimethylphenyl)-4,5-dihydroimidazol-2-ylidene (SIMes, NHC carbene), ethylenediamine, diethylenetriamine, tetramethylethylenediamine, aniline, pyridine, 2,2′-bipyridine, PPh₃ and PMe₃.

5. The process as claimed in claim 1, wherein the molybdenum in the molybdenum-based compound has an oxidation state of zero.

6. The process as claimed in claim 1, wherein the catalyst system comprises an additional Lewis acid.

7. The process as claimed in claim 6, wherein the Lewis acid is cationic or charge-neutral.

8. The process as claimed in claim 7, wherein the Lewis acid is cationic.

9. The process as claimed in claim 8, wherein the cationic Lewis acid is an unsubstituted dicyclopentadienyl metal cation, a substituted dicyclopentadienyl metal cation, an unsubstituted metal porphyrin cation, a substituted metal porphyrin cation, an unsubstituted metal salen cation, a substituted metal salen cation, an unsubstituted metal salphen cation and/or a substituted metal salphen cation.

10. The process as claimed in claim 8, wherein the molybdenum-based compound is anionic.

11. The process as claimed in claim 1, wherein the catalyst system has the structure (I), (II), (III), (IV), (V), (VI) and/or (VII): 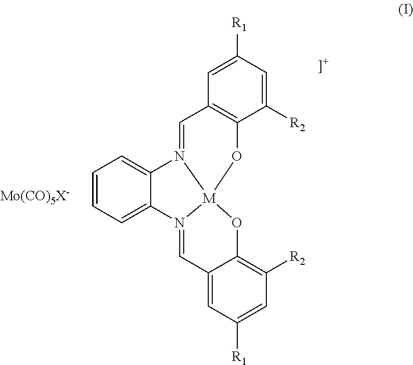
where X═H, F, Cl, Br, I, CN, NC, SCN, NCS, CP, N₃, NO₂, NO₃, NH₂, OTf, OH or HSO₄.
where M=Cr(III), Al(III), Fe(III), Co(III), Mn(III), V(III), In(III), Ga(III), Y(III), Ru(III), La(III), Ce(III), Gd(III) or Ir(III);
where R₁ and R₂ are each independently selected from the group consisting of hydrogen (—H), methyl, tert-butyl, phenyl, nitro, bromine, chlorine, hydroxyl, diethylamino and methoxy; 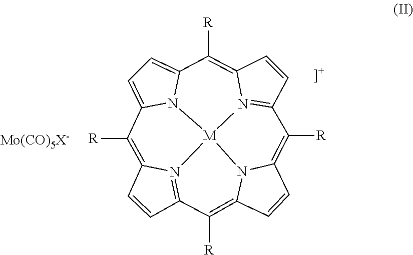
where X═H, F, Cl, Br, I, CN, NC, SCN, NCS, CP, N₃, NO₂, NO₃, NH₂, OTf, OH or HSO₄;
where M=Cr(III), Al(III), Fe(III), Co(III), Mn(III), V(III), In(III), Ga(III), Y(III), Ru(III), La(III), Ce(III), Gd(III) or Ir(III);
where R is selected from the group consisting of hydrogen (—H), methyl, tert-butyl, methoxy, phenyl, 4-methylphenyl, 4-methoxyphenyl, 4-chlorophenyl, 4-bromophenyl, 4-carboxylphenyl, 3,5-dimethoxyphenyl, 2-pyridyl, 4-pyridyl and N-methyl-4-pyridyl; 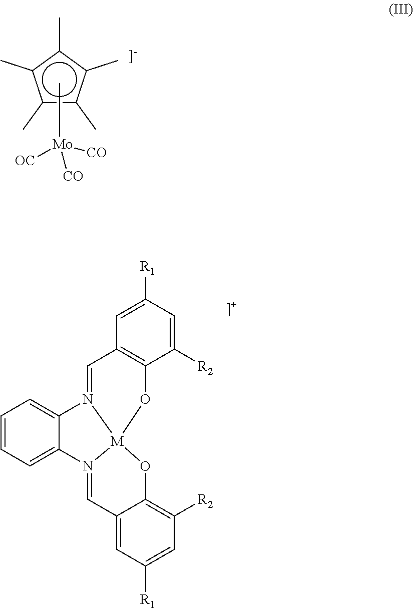
where M=Cr(III), Al(III), Fe(III), Co(III), Mn(III), V(III), In(III), Ga(III), Y(III), Ru(III), La(III), Ce(III), Gd(III) or Ir(III);
where R₁ and R₂ are each independently selected from the group consisting of hydrogen (—H), methyl, tert-butyl, phenyl, nitro, bromine, chlorine, hydroxyl, diethylamino and methoxy; 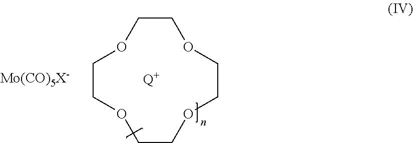
where X═H, F, Cl, Br, I, CN, NC, SCN, NCS, CP, N₃, NO₂, NO₃, NH₂, OTf, OH or HSO₄;
where Q=Li, Na, K, Rb, Cs, Cu or Ag;
where n=1-5 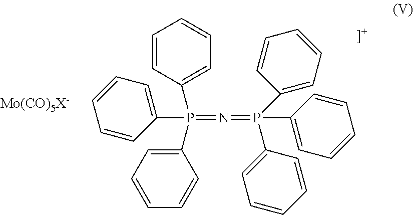
where X═H, F, Cl, Br, I, CN, NC, SCN, NCS, CP, N₃, NO₂, NO₃, NH₂, OTf, OH or HSO₄;
Q[Mo(CO)₅]; (VI)
where Q=Li, Na, K, Rb, Cs, Cu or Ag;
and/or 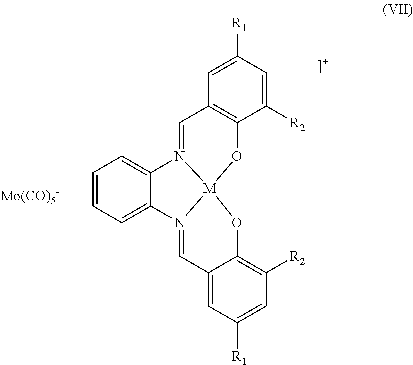
where M=Cr(III), Al(III), Fe(III), Co(III), Mn(III), V(III), In(III), Ga(III), Y(III), Ru(III), La(III), Ce(III), Gd(III) or Ir(III);
where R₁ and R₂ are each independently selected from the group consisting of hydrogen (—H), methyl, tert-butyl, phenyl, nitro, bromine, chlorine, hydroxyl, diethylamino and methoxy.

12. The process as claimed in claim 1, wherein the catalyst system has the structure (VIII), (IX), (X), (XI), (XII), (XIII), (XIV), (XV) and/or (XVI):

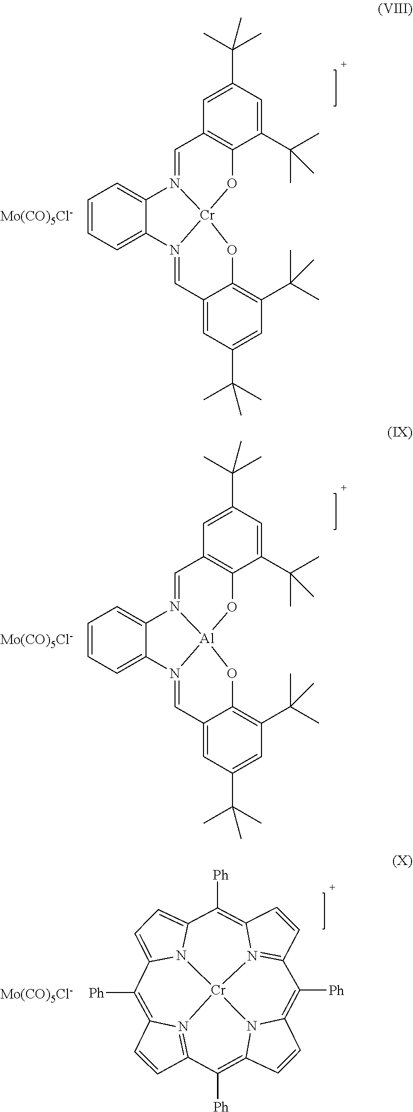

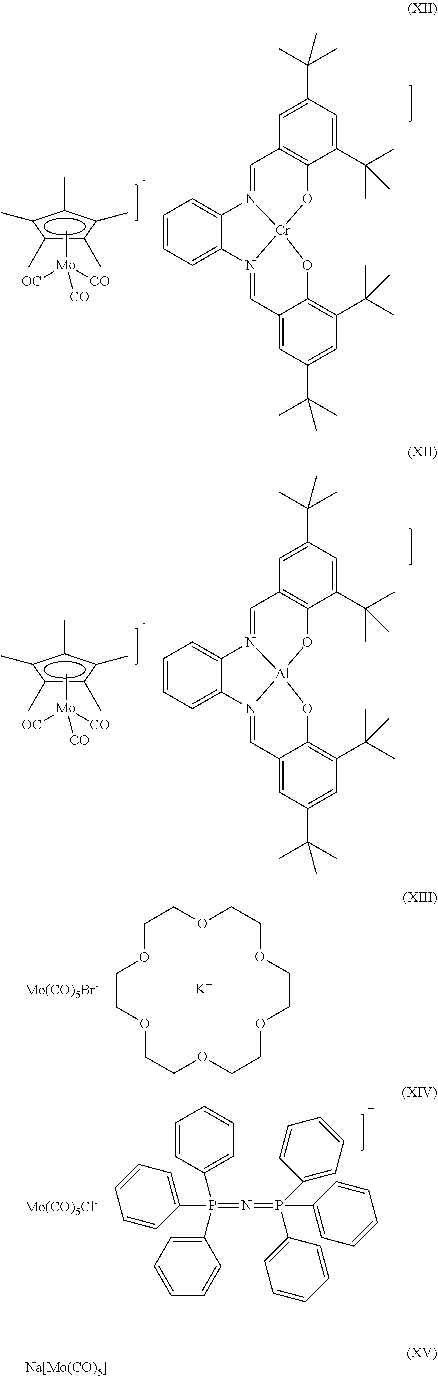

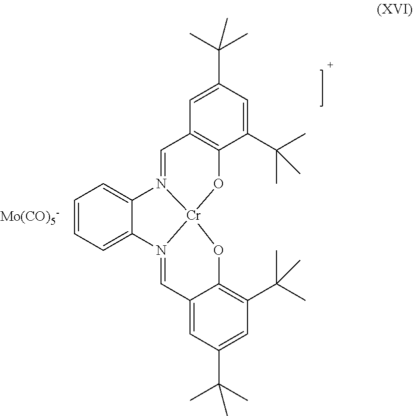

13. A carbonylation product obtainable by a process as claimed in claim 1, wherein the molar proportion of cyclic anhydrides, based on the epoxide used, is less than 5 mol % determined by ¹H-NMR.

14. A process for producing carbonylation conversion products by reacting the carbonylation products as claimed in claim 13 with epoxides, polyisocyanates and/or polycarboxylic acids.

15. A method comprising carbonylation of epoxides using the catalyst systems comprising a molybdenum-based compound as claimed in claim 1.

16. The process as claimed in claim 2, wherein the molybdenum-based compound comprises two to five carbonyl ligands.

17. The process as claimed in claim 4, wherein the ligand (L) is one or more compounds selected from the group consisting of Cl, Br and pentamethylcyclopentadienyl (Cp*, η⁵-C₅Me₅).

18. The process as claimed in claim 7, wherein the Lewis acid is cationic.

19. The process as claimed in claim 14, wherein the carbonylation conversion products are polyurethanes.

20. The process as claimed in claim 14, wherein the carbonylation products are reacted with polyisocyanates.
